(19) United States
(12) Patent Application Publication
(10) Pub. No.: US 2003/0216770 A1
(43) Pub. Date: Nov. 20, 2003

(54) Apparatus and method for making a percutaneous access port of variable size

(76) Inventors: Maxim D. Persidsky, Berkeley, CA (US); Andre M. Persidsky, San Francisco, CA (US)

Correspondence Address: Andre Persidsky, 1537 Jones Street, #202, San Francisco, CA, 94109, US

(21) Appl. No.: 10/371,508

(22) Filed: Feb. 20, 2003

Related U.S. Application Data
(60) Provisional application No. 60/358,774, filed on Feb. 21, 2002.
(60) Provisional application No. 60/435,116, filed on Dec. 19, 2002.

(51) Int. Cl. A61M 29/00

(52) U.S. Cl. 606/191; 606/198

(57) ABSTRACT

A device for creating a percutaneous access port of variable size through dilation of an initial percutaneous penetration in a body cavity wall. The device includes an insufflation and access needle for producing an initial puncture within the body cavity wall, a tubular dilator having an axial length, distal end for insertion within the body cavity wall and a proximal end for extending outside the body cavity wall during creation of the percutaneous access port. The tubular dilator has a single piece tubular body having a stem section between its distal and proximal ends and an expanded funnel-shaped surface at its proximal end. The tubular dilator can be releasably attached to the insufflation and access needle at its distal end into an axial compression device at its funnel-shaped proximal end. The axial compression device can apply reversible axial compression force upon the tubular dilator to change its axial length resulting in selective and reversible radial dilation of the device and resulting puncture.

### RELATED APPLICATIONS

The present application is based upon provisional U.S. application Ser. No. 60/358,774 filed on Feb. 21, 2002 and provisional U.S. application Ser. No. 60/435,116 filed on Dec. 19, 2002.

### TECHNICAL FIELD OF INVENTION

The present invention relates generally to an apparatus and method used in performing minimally invasive surgical procedures for accessing the abdominal and thoracic body cavities. More particularly, the present invention relates to an improved apparatus and method for creating a percutaneous access port where the initial penetration made by an insufflation and access needle is enlarged. Furthermore, the present invention relates to a new apparatus, which permits safe and easy formation of a percutaneous access port of variable size by means of radial dilation of an initial percutaneous penetration, utilizing a force generated through compression of a stretched out braided wire mesh of special configuration in accordance with the present invention.

### BACKGROUND OF THE INVENTION

With rapid advances in modern medicine, the application of laparoscopic and other minimally invasive surgical procedures has greatly increased. There is also a rapid increase in the complexity of these procedures and in the need to deploy larger and more numerous trocars. Consequently, the number of trocar-related injuries and complications currently is also on the rise. Such injuries and complications may include major vascular injury, bowel perforation, trocar wound site bleeding, post operative incisional hernias, intrapertoneal hemorrhage, and other vascular gastrointestinal and urogenital complications. As a result, there is a growing urgency to improve trocar safety.

Several approaches have been developed to reduce trocar-related injury. Some disposable trocars are now equipped with a retractable external tubular shield which rapidly covers the sharp trocar tip after it enters the peritoneal cavity. However, in spite of such shielding, a serious injury may still occur before the shield is fully deployed. A modification of this approach is used by the Dexide trocar, which uses an internal safety shield with its Woodford spike. Shaped like the tip of a large hypodermic needle, the Dexide trocar spike has a spring-loaded plastic plug inside, which shields its sharp tip and cutting edges. During penetration, this plug retracts, exposing the cutting tip and sharp edges of the spike, and then, after intraperitoneal entry, springs back into its initial shielded position. Although it may be safer than the external trocar shield, according to Dexide company claims, it is still based on a similar principle, which is not completely injury proof. Also, these shielding techniques do not address the danger associated with the relatively large diameters of most trocars, and the resulting wound size.

An entirely different way to resolve the trocar-related safety problem was pursued by InnerDyne, Inc. (recently acquired by United States Surgical), disclosed in U.S. Pat. Nos. 5,183,464, 5,431,676, and 6,080,174. Since their approach is relevant to the present invention, it will be discussed here in some detail and in an appropriate perspective.

In 1994, InnerDyne, Inc. introduced a new disposable laparascopic device under the trade name Step™ for forming and enlarging a percutaneous penetration, which was followed in 1996 by its more cost-effective version called the Reposable Step™ system. The operation of both devices is based on the idea of using radial dilation to enlarge a small puncture track made initially by an insufflation access needle through the abdominal wall. This is accomplished by utilizing an expandable tubular braid, introduced into this needle track in a radially compressed state, and then expanded by forcefully inserting an elongated blunt tapered dilator. This expansion mechanism makes it possible to avoid the use of large, sharp trocar cutting blades and the associated risk of injury.

The idea of dilating tissue by radial expansion with a tubular braid generally is not new. It already appeared in the mid 1980's with the introduction of the Urolum WallStent, disclosed in U.S. Pat. No. 4,655,771, as an expandable endoprostesis initially for endovascular use. Then its use was extended for dilation of the urethra, which was followed by enteral, tracheal, bronchial, and other applications. The tubular braid, made of biocompatible surgical grade super-alloy filaments, was first manufactured for such medical use in Switzerland by Schneider Co., and now is produced by Schneider (USA) Inc.

Another expandable stent of slightly different construction—made of titanium—was introduced at about the same time by Advanced Surgical Instrument Company for placement in the urethra.

The expandable stents from both of these companies are deployed endoluminally in a radially compressed state using special deployment tools.

The Urolum WallStent is initially loaded in a compressed, stretched state into a narrow tubular deployment tool, which is placed into the urethra. When the stent is released from this tubular tool, its resilient braided mesh expands radially on its own to its unconstrained diameter, thereby dilating the urethra.

The Inter-Prostate stent, which is non self-expanding, is mounted over an elongated narrow noncompliant balloon, which is inserted into the urethra and then inflated to expand the stent, thus dilating the urethra. After deflation, the balloon is withdrawn, and the stent provides enough outward radial force to maintain the patency of the urethra.

By extending the idea of radial dilation of tissue using a tubular braid for the formation of a laparascopic access port, InnerDyne, Inc. provided a much safer way of making a percutaneous access port with their Step devices, than present trocars can offer. Since the self-expansion force generated by the compressed braided sleeve is insufficient to dilate the abdominal wall, the dilation by the Step devices is accomplished by forcing through the braided sleeve a series of blunt obturators, one at a time, in progressively increasing diameters. Each of these dilators is positioned inside a matching size cannula from which only the tapered blunt end of the dilator emerges. Following radial expansion of the braided sleeve and dilation of the surrounding tissue, the dilator is removed, leaving the cannula barrel inside the braided sleeve. This cannula-based approach provides the internal support to the braided sleeve for maintaining it in an expanded state, and serves to provide a fixed radius working access port into the endoperitoneum.

Anchoring of the braided sleeve/cannula assembly to the abdominal wall relies mainly on the traction between the braided sleeve and surrounding tissues stretched by the expansion of the braided sleeve. This traction, according to InnerDyne, Inc., is sufficiently strong to eliminate cannula slippage, and hence loss of pneumoperitoneum.

The cannula is equipped at its proximal end with a removably attached valve for preventing the loss of pneumoperitoneum during installment of the cannula and introduction of surgical instruments, or withdrawal of tissue samples.

To select the desired size of the access port to be made by the Reposable Step device, or to step up its size during the procedure, several kits with components and replacement parts of different sizes are provided. They include radially expandable braided sleeves of 5, 7, 8, 10, and 12 mm diameters with matching valves, cannula assemblies, and dilators, each in 5, 7, 8, 10, and 12 mm diameters, and some additional parts.

In more detail, the procedural steps for making the percutaneous access port for laparascopic use, employing the Reposable Step system, consists first of puncturing the abdominal wall at the selected surgical site with an insufflation and access needle. Then, following insufflation, the needle is removed and inserted into the radially expandable sleeve. To prevent loss of pneumoperitoneum through the needle puncture track, InnerDyne, Inc. recommends covering this track with the surgeon's finger until the needle/sleeve assembly is reinserted into the same track. After reinsertion, the needle is withdrawn, leaving the braided sleeve in place. Again, to prevent the loss the pneumoperitoneum, through the open end of the braided sleeve, it must also be covered by a finger until one of the dilator/cannula devices, with a valve attached to its proximal end, is inserted into the braided sleeve. The tapered blunt dilator/cannula is gently pushed through the braided sleeve using a twisting motion, expanding it and radially dilating the track. Finally, the dilator is removed, leaving the braided sleeve with the cannula and valve in place, thus providing a completed endoscopic access port in a working position. If a larger port is needed during the procedure, the present port can be enlarged by replacing the smaller cannula with a larger cannula/dilator assembly, which is reinserted into the same expandable sleeve. Once again, during this rearrangement, the braided sleeve without the cannula and valve must be kept sealed by the finger of the operator. Furthermore, when the cannula is removed, the braided sleeve loses internal support and may collapse. As a result, the braided sleeve can lose traction with the surrounding tissue and hence, its anchoring capacity. In a worst case scenario, this may cause dislodgement of the sleeve, rapid loss of the pneumoperitoneum, interruption of the surgical procedure, and injury to the internal organs of the patient by other cannulas of other ports impinging on them, or by instruments present in some of the cannulas. Care must be exercised to prevent this from happening.

In spite of many drawbacks in the design of the InnerDyne laparoscopic access system—and the complexity of its operational procedure involving the use of many accessory parts—it offers important advantages that current trocars cannot provide.

The radial dilation of the insufflation and access needle entry track by the Step and Reposable Step devices tamponages blood vessels, producing virtually blood-free access. After the Reposable Step device is removed from the abdominal wall, the defect in each muscle layer contracts, leaving a series of non-overlapping slits, with a fascial defect less than half the size of that produced by sharp trocars. The small size of this defect usually eliminates the need for fascial closure. It also greatly reduces the risk of an incisional hernia.

Considering the importance of these advantages, it would be of great value to improve this system by eliminating its previously mentioned deficiencies, while building on its main positive feature—the radial dilation of tissue—to provide a new more efficient and practical apparatus for producing percutaneous access ports. This is the main object of the present invention.

In general, it is an object of the present invention is to provide an improved apparatus and method for facilitating safe and easy formation of percutaneous access to the abdominal and other body cavities, required in performing minimally invasive surgical procedures.

More specifically, a further object of the present invention is to allow formation of a percutaneous access port with minimal trauma by first making a small puncture through the wall of the body cavity with an insufflation and access needle, and then enlarging this puncture track by employing a new expansion mechanism, based on gradual radial dilation, using an innovative tubular dilator.

Furthermore, an object of the present invention is to provide an efficient apparatus designed largely around the incorporation of the novel tubular dilator, and containing the proprietary means for the dilator's expansion, thereby greatly simplifying the formation of the percutaneous access port.

Particularly, an object of the present invention is to provide a new apparatus with multifunctional capabilities, allowing penetration, insufflation, dilation, and the anchoring functions, all with one uninterrupted action, using a single device.

Moreover, an additional object of the present invention is to provide an apparatus that can form a variable size percutaneous access port for the introduction of surgical instruments in a wide range of sizes, and to provide a percutaneous access port that subsequently can be reduced in size for safe and easy withdrawal of the apparatus from the patient's tissue.

Yet another object of the present invention is to provide firm and secure anchoring of the access port, by forming an expansion clamp at the distal end of the tubular dilator of the new design, while accomplishing this with the same action that forms the access port.

Still another object of the present invention is to provide an improved adjustable pneumostatic valve to accommodate passage of variable size surgical instruments, and to prevent the catching of tissue samples as they are withdrawn through this valve.

Finally, a further object of the present invention is to provide an easy to use and inexpensive apparatus consisting of a single disposable unit, which can simplify the efforts of making a percutaneous access port, reduce the operation time and cost, and most importantly, to maximize the safety of the overall procedure.

These and other objects of the present invention will be apparent from the drawings and detailed description herein.

### SUMMARY OF THE INVENTION

According to the present invention, an improved apparatus and method for making percutaneous access ports are provided. The apparatus of the present invention eliminates the need to use separate devices for penetration, insufflation, dilation, and anchoring-all these functions are performed using a single percutaneous access device and are accomplished by a continuous and uninterrupted action.

The multifunctional capability of this new device is achieved by integrating in its design several functionally important components, the first of which is an expandable tubular dilator of novel proprietary design, which functions as a tissue dilator, anchor, and variable size cannula. In its first embodiment, the tubular dilator is made from wire mono filaments of stainless steel, nitinol, or other suitable alloy, or of a suitable polymer such as polyamide, braided into a tubular mesh. The mechanical principle on which the tubular dilator operates becomes apparent from the kinematics of its geometry. Each mesh of the tubular dilator forms a small equilateral parallelogram (rhombus), which, in accordance with the toggle-joint mechanical principle, displays force-amplifying behavior as it changes shape during axial compression of the braid from a sharp to flat angle along this axis. A well known practical application of this principle can be seen e.g. in a scissor jack or toggle-joint press. As the tubular dilator is axially compressed and shortened, and its parallelogram's opposite angles lying along this axis become greater than 90 degrees, the compound radial expansion force generated by all of these parallelograms becomes greater than the axial compression force applied to the braid. The gain in the expansion force and the dilator diameter both reach a maximum when the opposite angles of the parallelograms approach 180 degrees, generating sufficient force for radial dilation of tissue to create a working percutaneous channel of variable size. The second embodiment of the tubular dilator is made from a tube having a laser cut pattern of a matrix of connected parallelograms, weakened at their joints to allow them to radially expand upon axial compression of the tube also in accordance to the toggle-joint principle. This tube is made from stainless steel, nitinol, or other suitable alloy.

The second important functional component of the percutaneous access device of the present invention provides the means for making the initial percutaneous penetration and insufflation. It comprises an elongated hollow stylet, containing the insufflation and access needle. The stylet has a tapered cap at its distal end, which is open for releasing the needle. The cap also has a tubular cover attached to its proximal end. The distal end of the tubular dilator is compressed and fitted around the stylet under its very thin walled tubular cover from the proximal side of the cap, where it is held by tension. The proximal end of the stylet has a spring-loaded catch for controlling the step-wise release of the needle length from the open cap at the distal end of the stylet.

The third important functional component of the percutaneous access device is the means of generating the axial compression force on the tubular dilator to effect its radial dilation. Three embodiments have been developed, each employing a different structure to generate this force, but all utilizing a pair of pulling wires attached near the distal end of the dilator for transmitting the force. This structure for providing the axial compression is detailed for the first embodiment in the procedural summary below.

The procedure for using the percutaneous access device of the present invention is very similar for all three embodiments. It first requires measuring the thickness of the abdominal wall (in laparoscopic application) for appropriately presetting the needle length advancement from the stylet. Then, a special new device for the non-invasive lifting of the abdominal wall can be optionally applied on the abdomen at the surgical site in order to provide a small clearance in the abdomen for safer initial needle insertion. Thereafter, the percutaneous access device is moved forward until the insufflation and access needle passes through the abdominal wall, and the tip of the stylet cap touches the skin. This positions the needle correctly for insufflation. After insufflation of the abdomen, the needle is partially retracted back into the stylet while its tip remains outside the stylet cap and within the puncture track. Next, the needle tip along with the stylet cap are both pushed forward through the same track until the proximal end of the cap is flush with the skin. At this point, the needle is completely retracted into the stylet for safety, and the cap is further advanced into the abdominal cavity until the base of the funnel of the tubular dilator is flush with the skin. Now the tubular dilator is correctly positioned for expansion. Next, the distal end of the tubular dilator is released from the tubular cover, so that it can be expanded. This release and expansion is achieved by turning a knurled nut positioned around the housing of the percutaneous access device. Utilizing two pulley mechanisms inside the percutaneous access device, turning the nut causes two wire loops attached on opposite sides of the distal rim end of the tubular dilator to pull its end out from the cover on the cap. Turning the nut further causes the wire loops to apply more axial compression, thus radially expanding the tubular dilator more, and allowing the stylet together with the needle assembly to be removed from the dilator, and entirely from the percutaneous access device. To complete formation of the percutaneous access port, the tubular dilator, positioned at this point within the initial percutaneous channel, is radially expanded further, thereby dilating the channel to a desired size, allowing the required surgical instruments to pass through the device and channel. At this point, an expansion clamp is concurrently formed at the distal end of the tubular dilator, firmly anchoring the dilator along with the percutaneous access device in place for the procedure. During the procedure, if additional insufflation pressure is needed, gas is administered through an insufflation valve. Depending on the size of the surgical instruments needed, a cap with a small gasket can be unscrewed from the top of the percutaneous access device, allowing larger diameter instruments to be inserted. At the end of the surgical procedure and after removal of all surgical instruments, gas is released from the insufflation valve. Then the housing nut is turned completely in the opposite direction to collapse the tubular dilator to its initial narrow width, allowing the percutaneous access device along with the tubular dilator to be safely removed from percutaneous channel. The procedural steps for using the other two embodiments are very similar.

### BRIEF DESCRIPTION OF THE FIGURES

FIGS. 1A-C are side views of the initial braided cylinder, template, and final form for use in practicing the present invention.

FIGS. 2A-C are side views of alternative construction steps to fabricating the braided cylinder for use herein.

FIG. 3 is an enlarged side view showing in detail the attachment of a pulling wire loop to the rim of the flared distal end of the tubular dilator of the present invention.

FIG. 4 is a side view, partly in section, of the present expanded tubular dilator, forming an expansion clamp, which anchors it to a body cavity wall and also showing one of the pulling wire loops.

FIG. 5 is a side view of the housing cap holding a stylet at its center which contains an insufflation and access needle.

FIG. 5A is a top sectional view taken along lines 5A-5A of FIG. 5 partly in perspective of a spring-loaded stop.

FIGS. 6-6A are an enlarged perspective view, partly in section, of the preferred embodiment of the mechanism for holding in place and then releasing the stretched out end portion of the tubular dilator at the distal end of the stylet.

FIGS. 7-7A are an enlarged perspective view, partly in section, of an alternate embodiment of the means for holding in place the stretched out end portion of the tubular dilator at the distal end of the stylet.

FIG. 8 is a sectional view, partly in perspective, of the first embodiment of the percutaneous access device of the present invention.

FIG. 9 is a sectional view, partly in perspective, of the first embodiment of the percutaneous access device of the present invention, with the present tubular dilator released from the covering cap of the stylet.

FIG. 10 is a sectional view of the first embodiment of the percutaneous access device of the present invention, fully installed in a percutaneous channel and with the stylet removed.

FIG. 11 is a sectional view along line 11-11 in FIG. 10.

FIG. 12 is a sectional cutaway view, partly in perspective, of the first embodiment of the percutaneous access device of the present invention, rotated 90 degrees with respect to FIG. 10 to show both pulleys for controlling axial compression of the dilator.

FIG. 13 is a sectional view along line 13-13 in FIG. 12.

FIG. 14 is a sectional view of the flapper portion of the pneumostatic valve of the present invention.

FIG. 15 is a perspective view of the flapper portion of the valve rotated 90 degrees with respect to FIG. 14.

FIG. 16 is an exploded sectional view of the removable gate portion of the pneumatic valve of FIG. 14.

FIG. 17 is an exploded sectional view of the non-removable gate portion of the pneumatic valve of FIG. 14.

FIG. 18 is an enlarged perspective view of the percutaneous access device before its use.

FIG. 19 is a perspective view of the percutaneous access device in its closed initial position.

FIG. 20 is a perspective view, partly in section, of the percutaneous access device in its insufflating position with its insufflation and access needle appropriately extended.

FIG. 21 is a perspective view, partly in section, of the percutaneous access device, with the tapered cap of its stylet pushed through the body cavity wall with the insufflation and access needle partially retracted.

FIG. 22 is a perspective view, partly in section, of the percutaneous access device, with its tubular dilator fully installed before expansion in the abdominal wall.

FIG. 23 is a perspective view, partly in section, of the percutaneous access device with its tubular dilator released from its tapered cap and showing an expanded flare at its distal end.

FIG. 24 is a perspective view, partly in section, of the percutaneous access device with its tubular dilator partially expanded to 8 mm and the stylet removed.

FIG. 25 is a perspective view, partly in section, of the percutaneous access device with its tubular dilator further expanded to 10 mm.

FIG. 26 is a perspective view, partly in section, of the percutaneous access device with its tubular dilator further expanded to 12 mm.

FIG. 27 is a perspective view, partly in section, of the percutaneous access device with its tubular dilator collapsed to its initial size for removal from the body cavity wall.

FIG. 28 is a sectional view, partly in perspective, of a second embodiment of the percutaneous access device of the present invention.

FIG. 29 is a perspective view of a portion of the second embodiment of the percutaneous access device of the present invention, illustrating its position dial and pointer.

FIG. 30 is a sectional view along line 30-30 in FIG. 29.

FIG. 31 is an exploded view of the gear train of the second embodiment.

FIG. 32 is a sectional view of a tubular pin used to fasten both ends of a pulling wire to one of the rollers.

FIG. 33 is a sectional view, partly in perspective, of the third preferred embodiment of the percutaneous access device of the present invention in its initial position.

FIG. 34 is a sectional view, partly in perspective, of the third preferred embodiment of the percutaneous access device installed in an abdominal wall.

FIG. 35 is a perspective view, partly in section, of the third preferred embodiment of the percutaneous access device installed in the abdominal wall and showing a numerical scale.

FIGS. 36-38C are various views of the second laser cut embodiment of the tubular dilator.

FIGS. 39-41 are side sectional views of the funnel connector attachment to the percutaneous access device.

FIGS. 42-46 are various, plan and prospective views of the alternative means of attaching pulling wires to the funnel dilator of the present invention.

FIG. 47 shows a way to link the wire ends of the mesh to prevent unraveling.

FIG. 48 is a side plan view of a preferred dilator for use herein where the intersection of wires proximate the distal end of the dilator are joined by use of flexible hinges.

FIGS. 49-52 are side plan view of the flexible hinges of FIG. 48 shown in expanded detail.

FIGS. 53-55 is a side perspective view of a further embodiment of an insufflation and access needle useful in practicing the present invention.

FIGS. 56A-57 are partial crossectional views of a preferred means of attaching control wires for altering the axial length of said dilator.

FIGS. 58-60 are side plan views of yet another embodiment of the present invention shown in performing the claimed method.

FIG. 61A is a sectional view of a templet used to fabricate the braided dilator.

FIG. 61B is a side view of a suitable tubular dilator derived from 61A.

FIGS. 61-64 show a sheath covering the tubular dilator.

### DETAILED DESCRIPTION OF THE INVENTION

The new tubular dilator of proprietary design plays a major role in the operation of the present percutaneous access device—it functions as a tissue dilator, cannula, and an anchor. It is made from mono filaments of stainless steel, nitinol, or other suitable alloy material, or from an appropriate polymer such as polyamide. In its first embodiment, the tubular dilator is made from a braided tubular wire mesh, compressed to 3 to 4 mm in diameter using a template, and approximately 50-75 mm long. Alternately, the first embodiment can be braided directly in its 3 to 4 mm diameter. The second embodiment of the tubular dilator is made from a tube having a laser cut pattern of a matrix of connected parallelograms, weakened at their joints to allow them to radially expand upon axial compression of the tube, made from stainless steel, nitinol, or other suitable alloy. When fully axially compressed, the tubular dilator will expand from approximately 3 mm to 14 mm, but can be made to expand to even larger diameters such as 20 mm provided that its initial diameter is made a little larger.

Each mesh of the wire braided embodiment of the tubular dilator forms a small equilateral parallelogram (rhombus), which, in accordance with the toggle-joint mechanical principle, displays force-amplifying behavior as it changes shape during axial compression of the braid from a sharp to flat angle along this axis. A well known practical application of this mechanical principle can be seen e.g. in a scissor jack or toggle-joint press. As the tubular dilator is axially compressed and its parallelogram's opposite angles lying along this axis become greater than 90 degrees, the compound radial expansion force generated by all of these parallelograms becomes greater than the axial compression force applied to the braid. The gain in the expansion force reaches a maximum when the opposite angles of the parallelograms approach 180 degrees. At the same time, the tubular dilator attains its maximum diameter. These kinematic changes in the braid geometry produce two important beneficial effects on the percutaneous access device function.

First, as the initial percutaneous penetration is dilated by radial expansion of the tubular dilator, there is a rising resistance of the tissue to increasing dilation. This resistance, however, is counteracted by the radial force generated through axial compression of the braid, which also increases as its width increases, along with the increase of the opposite angles of its parallelograms as described above. Such a direct relationship between these interactions provides an important mechanical advantage, allowing maximum dilation of the tissue with only a small increase in the axial compression force. Thus, radial expansion of the tubular dilator by axially compressing it offers a new powerful tool for dilating tissue, one that can be effectively used in forming percutaneous access ports of various diameters.

The second beneficial effect offered is that during dilation of the initial percutaneous channel passing across the wall of the body cavity, the distal stretched out portion of the tubular dilator extending into the body cavity expands more readily and to a larger diameter than the part constrained by the tissue of the wall. As a result, an expansion clamp having an hour glass shape is formed, which firmly anchors the tubular dilator along with the percutaneous access device assembly to the wall of the body cavity (e.g. the abdominal wall). Once this distal clamp is formed, the narrow part of the tubular dilator, constrained within the percutaneous channel, will continue to radially expand to the desired diameter and dilate the tissue, provided that there is an additional increase in the compression force.

The laser cut embodiment of the tubular dilator will experience similar kinematic changes in its laser cut parallelograms as the wire meshes of the braided tubular dilator embodiment upon axially compression, providing the same benefits to the function of the percutaneous access device.

Like the power amplifying scissor jack, the tubular dilator, with similar kinematics in its geometry, is also capable of translating a lesser input force of axial compression into a greater output force of radial expansion. By applying this kinematic principle to the dilation mechanism for enlarging an initial narrow percutaneous puncture, in accordance with the present invention, a major improvement in the technique for making percutaneous access ports of variable sizes is provided.

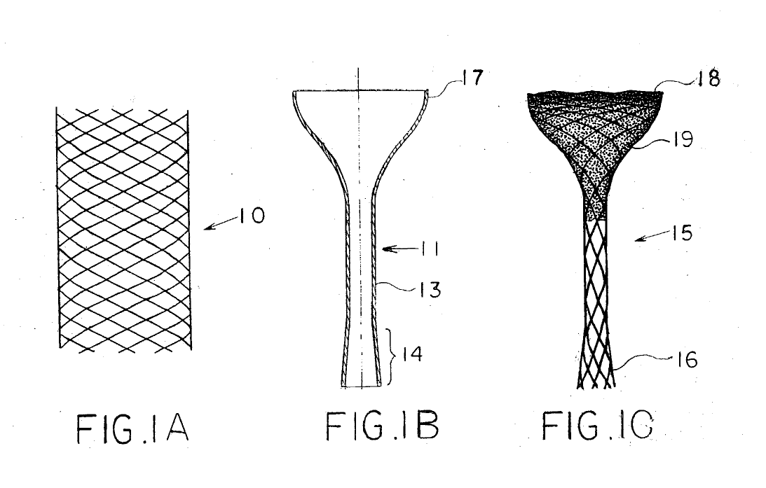

The adaptation of a tubular braid for use in the tissue dilating mechanism of the present invention, by modifying its shape and adding to it a pneumostatic seal, led to the funnel-shaped embodiment of the tubular braid, shown in FIG. 1C. Referred to as the tubular dilator, it provides the multifunctional capability to the percutaneous access device of the present,invention, which facilitates and simplifies making percutaneous access ports of variable sizes.

FIG. 1A shows a braided cylinder 10 made of stainless steel monofilament open wire mesh. Alternately, nitinol mono filaments can be used. It is approximately 50 to 75 mm long and is preferably 14 mm in diameter, with a wire diameter ranging from 0.005 inches to 0.2 inches, but preferably from 0.005 to 0.015 inches, although other sizes can also be employed for length, width, and wire diameter.

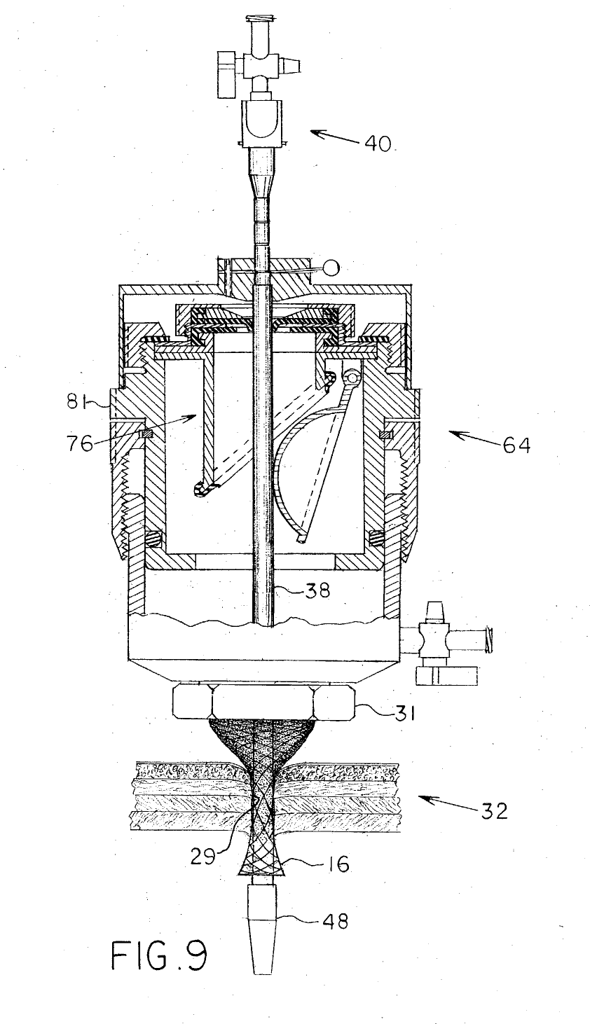

In order to modify braided cylinder 10 into the desired funnel-shape configuration, it is fitted into a special thin-walled metal templet 11 (FIG. 1B), having a conical distal end 14, a stem portion 13, and a broad proximal funnel end 17. Braided cylinder 10 in templet 11 is heat treated to make it conform to its new shape. Then braided cylinder 10 is removed from templet 11 in its final funnel-shape, referred hereinafter as tubular dilator 15 (FIG. 1C). As a result of the presence of conical distal end 14, a small outward flare 16 is formed on the distal end of tubular dilator 15, shown in FIG. 1C. The function of flare 16 will be described later. To prevent loss of pneumoperitoneum through the open mesh of tubular dilator 15, about half of tubular dilator 15, starting from its broad proximal end, is covered with a coating 19 of elastic material such as latex, Permalume, polyethylene C-flex, silicone rubber, or the like. The proximal rim 18 of tubular dilator 15 receives an additional denser coating functioning as a sealing gasket for the leak proof attachment of tubular dilator 15 by means of a nut 31 appended to the housing of a percutaneous access device 64 of the present invention as shown in FIG. 9. Alternately, the entire tubular dilator 15 can be covered with an elastic material such as latex, Permalume, polyethylene C-flex, silicone rubber, or the like to provide smoother percutaneous deployment of the dilator. One advantage of using braided cylinder 10 in its initially wider diameter is that it may be easier to braid more wire meshes circumferentially in the braid than if braided in a narrower configuration as described below.

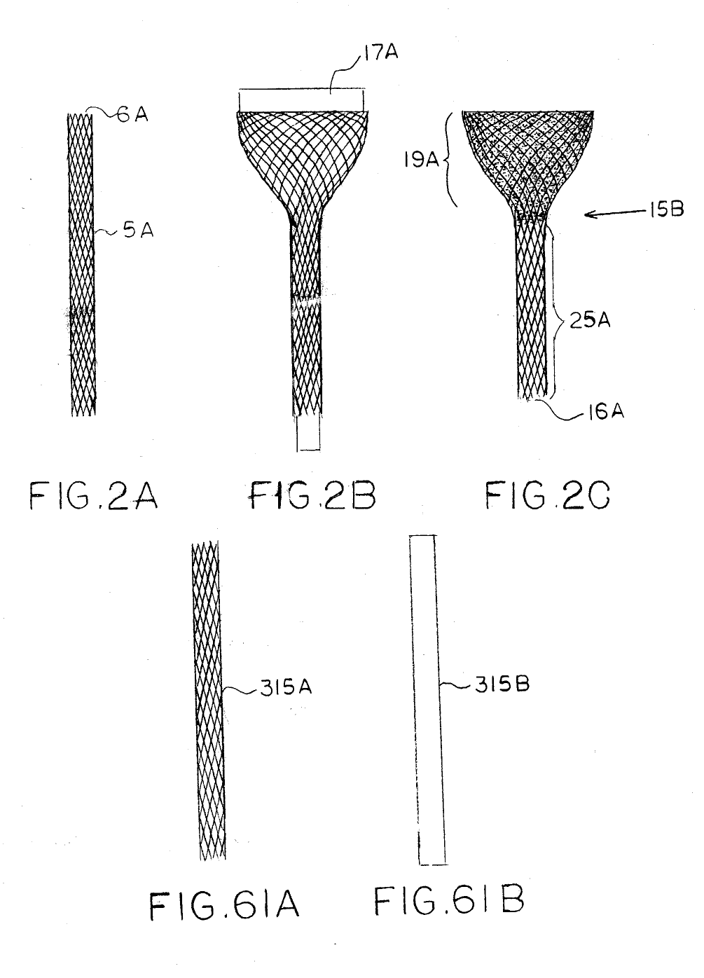

As shown in FIGS. 2A-2C, an alternate approach of making tubular dilator 15B without heat treating the material is by braiding it directly as a 3 to 4 mm wide tubular mesh 5A (FIG. 2A), cut into lengths of 50-75 mm, using stainless steel wire filaments 6A, approximately 0.005 to 0.015 inches in diameter, of a semi-springy stainless steel material. Tubular dilator 15B has a distal rim 16A, a stem section 25A, and a proximal funnel section 19A (FIG. 2C), which is created by outwardly stretching it over template 17A, and then coating approximately the top half of tubular dilator 15B with an elastic material such as latex, Permalume, polyethylene C-flex, silicone rubber, or another suitable elastic material to preserve its funnel shape, and also to serve as a seal to prevent the loss of pneumoperitoneum through its open mesh during surgery. Alternately, the entire tubular dilator 15B can be covered with an elastic material such as latex, Permalume, polyethylene C-flex, silicone rubber, or the like to provide smoother percutaneous deployment of the dilator. Alternately as well, tubular dilator 15B can be left entirely in its tubular form without stretching a proximal funnel, and connected to the percutaneous access device using a rubber funnel connector described later. Tubular dilator 15B can be created on a wire-braiding machine such as those supplied by OMA or Wardwell. At least 24 carriers are used to form at least 12 meshes circumferentially around tubular dilator 15B, but preferably at least 32 carriers or greater are used to form at least 16 meshes. The dilator is woven so that it is radially compressed in its resting state, approximately 3.5 mm in diameter. In order to radially expand from 3.5 mm to 12-14 mm when axial compression is applied, the number of pics or vertical meshes per inch for braiding is set relatively low so that the resulting wire meshes are narrow and vertically elongated when tubular dilator 15B is in its radially compressed state. This approach of making tubular dilator 15B does not utilize a flare at its distal end 16A.

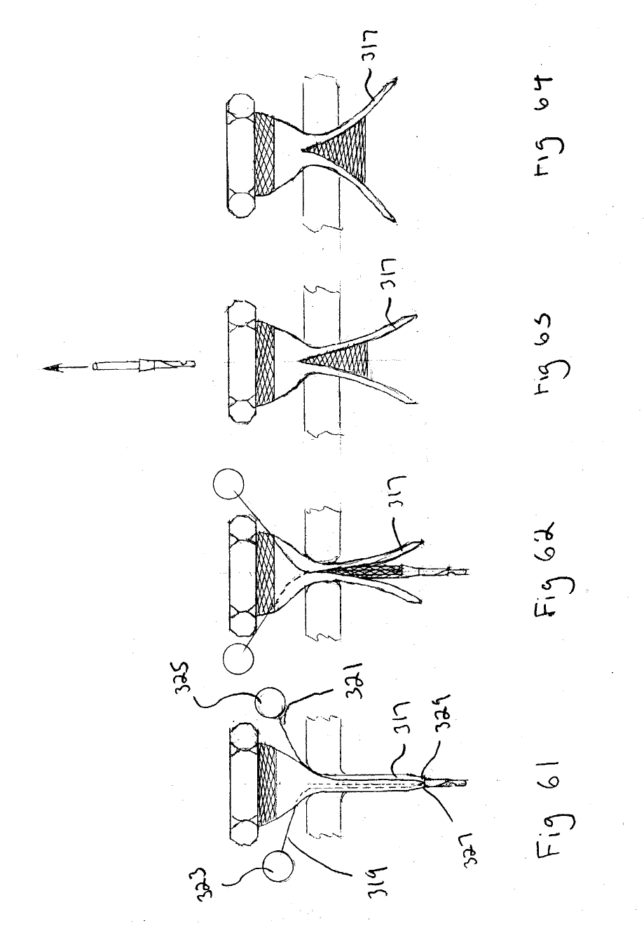

Tubular dilator 15/15B can be alternatively made from nitinol wire, making use of the superelastic properties of nitinol to make the dilator completely reversibly expandable, so that when the axial compression force or stress is removed, tubular dilator 15/15B will completely spring back to its original 3.5 mm diameter. In order to set the superelastic shape of tubular dilator 15/15B, it is heat treated after inserting into a narrow mandrel tube 315B (FIG. 61B), approximately 3 mm in diameter, if working with a nitinol tubular braid 315A braided in a 3 to 4 mm wide diameter as shown in FIG. 61A, or into template 11 if starting with a larger diameter nitinol braid as shown in FIG. 1A. Those skilled in the art of shape setting or training nitinol will know the appropriate temperature range and duration for heat treating, but generally between 400 and 500 degrees C. A special binary Nitinol alloy (available for example from NDC headquartered in Fremont, Calif.) is preferably used with an austenite finish temperature (Af) slightly below normal room temperature, so that when tubular dilator 15/15B is stress-induced into its radially expanded or martensitic form, tubular dilator 15/15B will subsequently revert back to its undeformed austenite narrow shape at room or body temperature immediately after the axial compression stress is removed. One advantage of using template 11 and a wider initial nitinol braid is that its proximal funnel shape and stem can be formed in one step, instead of using template 17A to stretch nitinol tubular braid 315A after being heat treated in mandrel tube 315B. Tubular dilator 15B in nitinol may be left entirely tubular as shown in FIG. 61A and attached to percutaneous access device 64 using a rubber funnel connector described later.

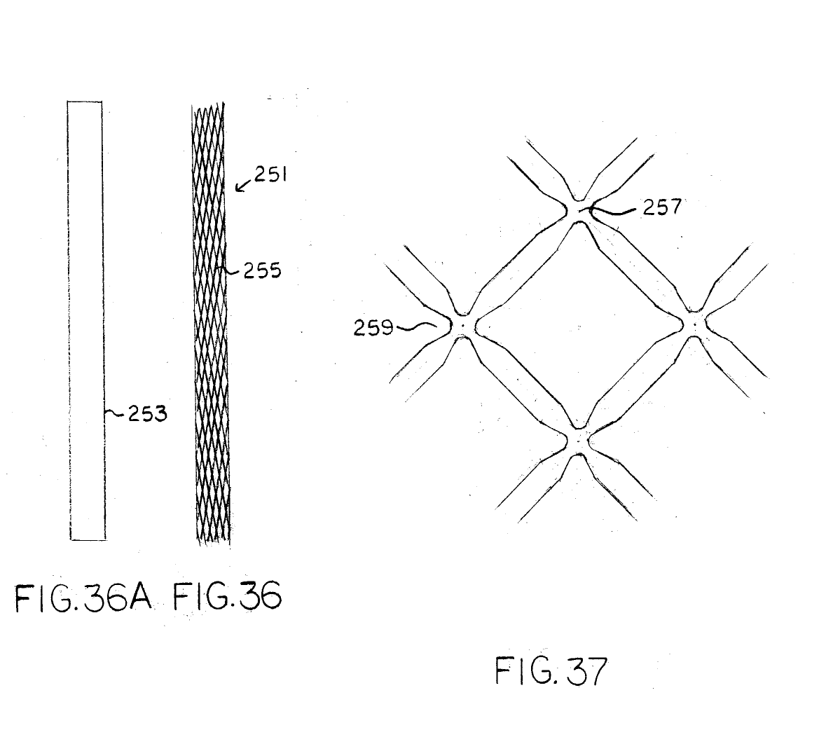

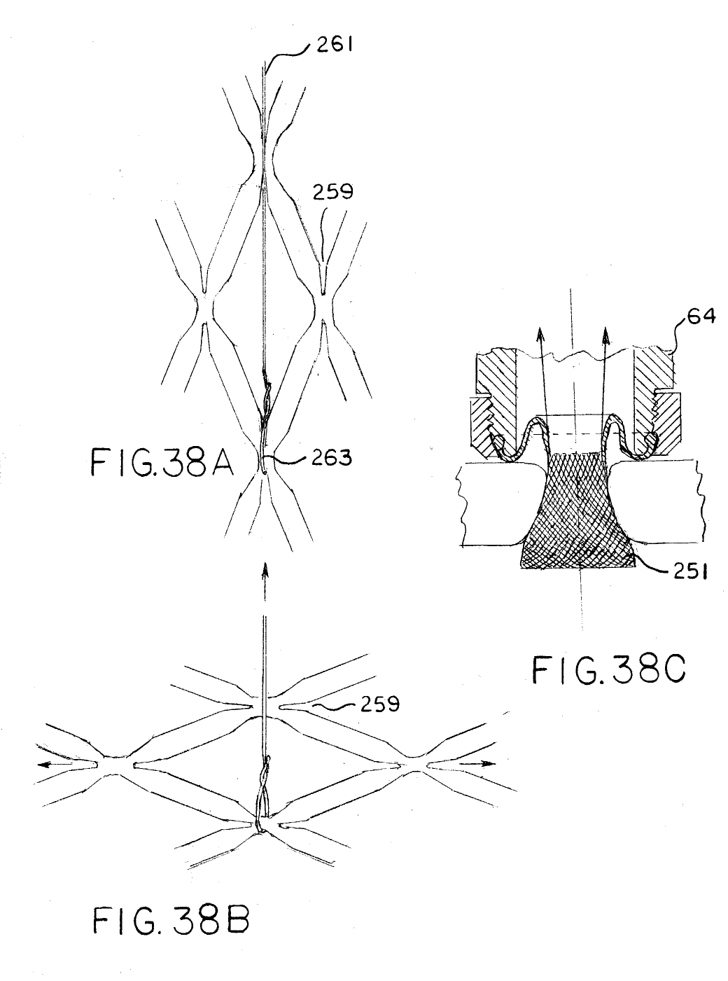

In FIGS. 36-38, the second embodiment of tubular dilator 251 does not utilize a braided wire mesh, but is instead made from a thin walled tube 253 having a laser cut pattern 255 consisting of a matrix of parallelograms, connected together at joining points. Tube 253 is made from stainless steel, nitinol, or other suitable alloy material. A joining point 257 is structurally weakened by laser cutting around it four small V shape cuts 259 which increases flexibility to allow joining point 257 to function as a joint so that when tubular dilator 251 is axially compressed, it will radially expand in accordance to the toggle-joint mechanical principle. When tubular dilator 251 is in its narrow unexpanded state (FIGS. 36 and 38A), V shape cuts 259 which are oriented along the vertical axis are in a closed V configuration and V shape cuts along the horizontal axis are in a open V configuration. As the dilator expands radially, the horizontally oriented V shape cuts 259 close and the vertically oriented V shape cuts open (FIG. 38B). In FIG. 38A, a pulling wire 261 is shown attached to a joining point 263 near the distal end of tubular dilator 251, to transmit the axial compression force. Pulling wire 261 is attached by looping, twisting, and soldiering its distal end around joining point 263. A similar pulling wire is also attached on the opposite side of tubular dilator. The pulling wires are described in greater detail in a later section. FIG. 38C shows tubular dilator 251 installed percutaneously. In order to attach tubular dilator 251 to percutaneous access device 64, its proximal end can be formed into a funnel shape and coated as previously described using a template, or it can be left tubular and attached using a rubber funnel connector as described below and shown in FIG. 38C. Tubular dilator 251 is preferably made from nitinol with superelastic characteristics at room and body temperature, such that tubular dilator 251 automatically springs back to its radially contracted state after the axial compression force is removed. The shape setting method for tubular dilator 251 is similar to that already described for the nitinol version of tubular dilator 15B. Tubular dilator 251 may be utilized in place of tubular dilator 15/15B in the three embodiments of the percutaneous access device disclosed herein. Tubular dilator 251 provides several important advantages over wire braided tubular dilator 15/15B. It's outside surface is smoother offering less friction during insertion and withdrawal from the percutaneous puncture. Being laser cut, tubular dilator 251 can be made with a higher degree of precision, and its radial expansion characteristics can be more precisely set. On the down side, tubular dilator 251 may be considerably more expensive to make than tubular dilator 15/15B. The entire tubular dilator 251 can be covered with an elastic material such as latex, Permalume, polyethylene C-flex, silicone rubber, or the like to provide even smoother percutaneous deployment of the dilator.

FIGS. 39-41 show an alternate way of attaching tubular dilator 15B/251 to the housing of percutaneous access device 64. In this variation, the dilator does not have a proximal funnel shape, but is left entirely tubular as shown in FIGS. 2A and 36. Instead, a funnel connector 265 made of rubber is joined to the proximal end of tubular dilator 15B/251 by molding the distal narrow end of funnel connector 265 into the metal matrix of the proximal end of tubular dilator 15B/251. Funnel connector 265 has a beaded ring 267 at its proximal end which snaps into a groove 269 at the distal end of percutaneous access device 64. A nut 271 with a conical rim 273 is then applied to permanently fasten funnel connector 265 to percutaneous access device 64 with rim 273 holding beaded ring 267 in groove 269. When tubular dilator 15B/251 is axially compressed and radially expanded, funnel connector 265 collapses inwardly into the distal end of percutaneous access device 64, so that the entire percutaneous access device 64 rests on the abdominal wall during surgery, cushioned by the rubber funnel connector 265. Funnel connector 265 also functions as a gas seal. This embodiment offers several advantages. The first is a reduction in trauma to the percutaneous puncture since its peripheral opening will have a smaller cross section, which also will help to anchor the device better. The second advantage is a simplified tubular dilator without having to form in it a funnel shaped proximal section.

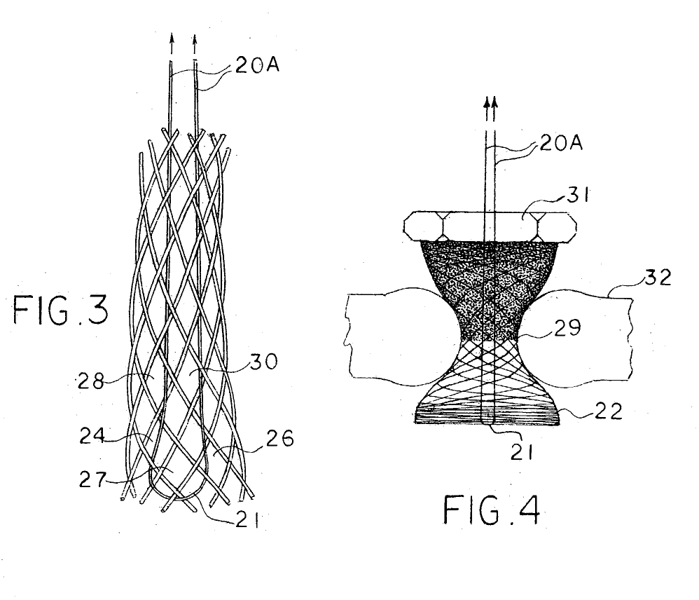

The axial compression and resulting radial expansion of tubular dilator 15/15B is accomplished by tension on a pair of pulling wires 20a and 20b (FIG. 6), attached on opposite sides of the distal end of tubular dilator 15/15B, by means of looping wires (20A and 20B, the details of which will be described hereinafter). Pulling wire 20A, and wire loop 21 are shown in FIG. 3 in an enlarged view of the front side of tubular dilator 15/15B. Tubular dilator 15/15B/251 is axially compressed relative to a stationary nut 31, as shown in FIG. 4, which mounts tubular dilator 15/15B/251 to percutaneous access device 64, as shown in FIG. 9. Pulling wire 20a is made from a high tensile strength titanium steel alloy, a high tensile strength stainless steel, or other high tensile alloy, allowing it to be thinner than the wire used in the mesh of tubular dilator 15/15B. Pulling wire 20a is attached to the distal end of tubular dilator 15/15B by first making a small wire loop 21, the long ends of which are then threaded outward through the last two wire mesh parallelograms 24 and 26 of tubular dilator 15/15B, which are separated by one wire mesh parallelogram 27 between them. After passing to the outside of tubular dilator 15/15B and over the two wire mesh parallelograms, the ends of pulling wire 20a are directed inward through its wire mesh parallelograms 28 and 30, from where they traverse all of the length of tubular dilator 15/15B, inside it in the proximal direction. Wire loop 21 is then woven and hooked between the wire ends of wire mesh parallelogram 27, at the distal end of tubular dilator 15/15B, where it remains firmly secured by constant tension exerted on pulling wire 20a. Pulling wire 20b on the opposite side of tubular dilator 15/15B is similarly attached (not shown). An alternate means of fastening wire loop 21 to the distal end of tubular dilator 15/15B is shown in FIG. 6.

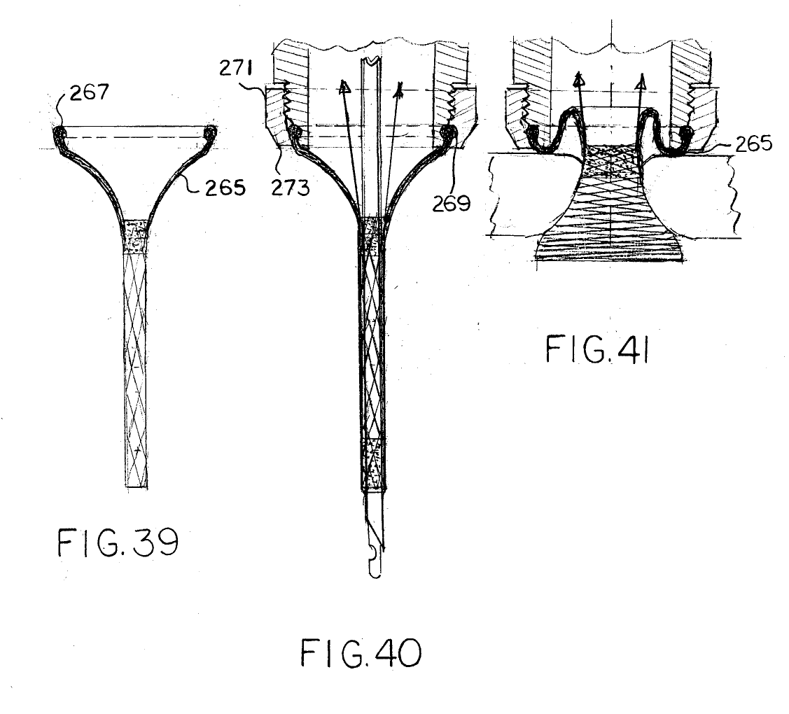

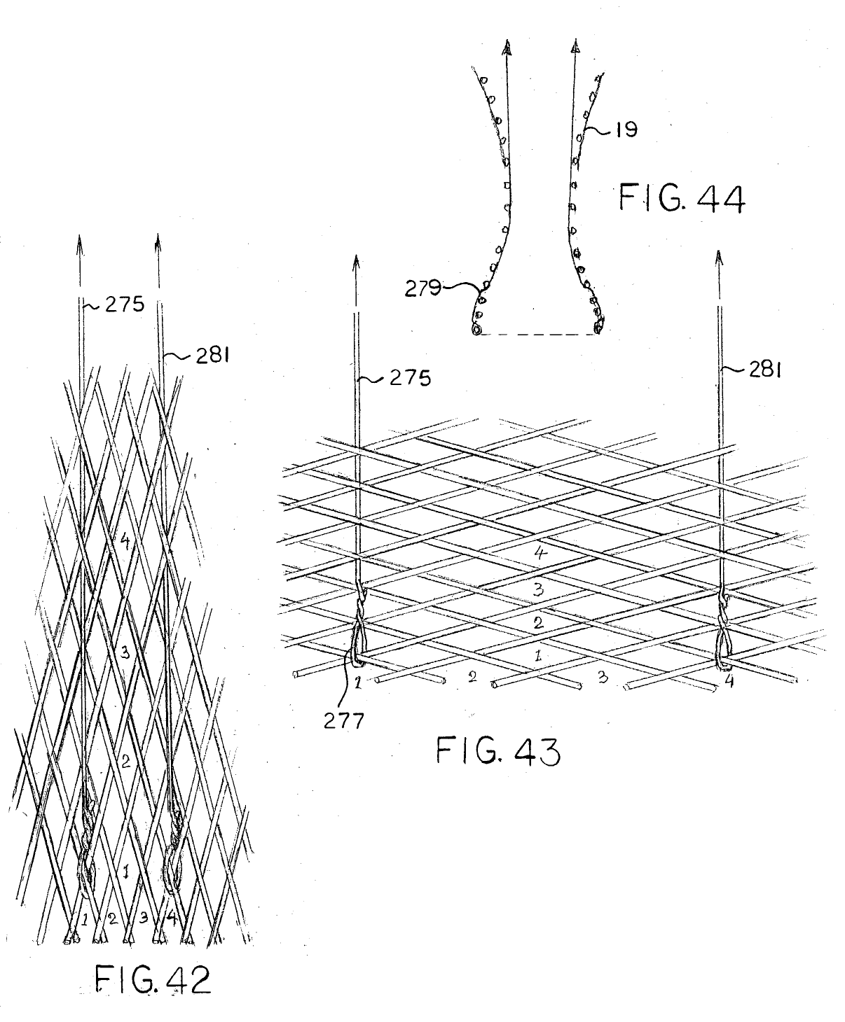

FIGS. 42-46 show alternate approaches of attaching pulling wires to the distal end of tubular dilator 15/15B. In FIGS. 42-44, a pulling wire 275 is used having a loop 277 at its distal end, which is formed by twisting and soldiering. Loop 277 is attached to a wire intersection of tubular dilator 15/15B. Pulling wire 275 runs proximally on the outside of tubular dilator 15/15B and is threaded into tubular dilator 15/15B at a third mesh 279 above its attachment point as shown in FIGS. 43 and 44. A similar wire 281 is attached at the same level but three meshes to the right. A similar pair of pulling wires is also attached on the opposite side of tubular dilator 15/15B. When a pulling force is applied to wires 275 and 281 (and the wires on the opposite side), they move apart as shown in FIG. 43, and cause the dilator to expand. This pulling wire arrangement will provide a uniform compression and radial expansion of tubular dilator 15/15B, and loop 277 will not interfere with the expansion of the mesh.

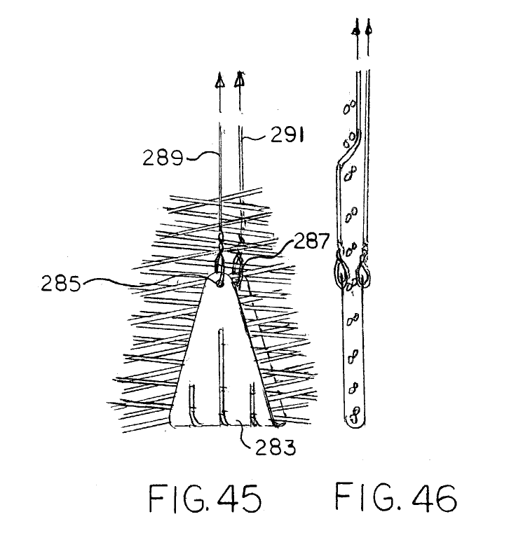

In FIGS. 45-46, a thin slotted ribbon 283 bent into a U shape is used to hug the distal rim of tubular dilator 15/15B. Ribbon 283 has holes 285 and 287 through which pulling wires 289 and 291 are (attached as shown. Pulling wire 289, which is attached to the side of ribbon 283 which is on the outside of tubular dilator 15/15B, runs proximally for three meshes, then into tubular dilator 15/15B through the fourth mesh as shown in FIG. 46, where it continues proximally and parallel to pulling wire 291. A similar ribbon with two pulling wires is attached 180 degrees apart on the distal rim of tubular dilator 15/15B to apply uniform compression to the dilator. The advantage of ribbon 283 is that it distributes the pulling force along the width of the ribbon, instead of the more point-like direct wire attachment. Ribbon 283 can be made from steel, plastic, or other suitable materials.

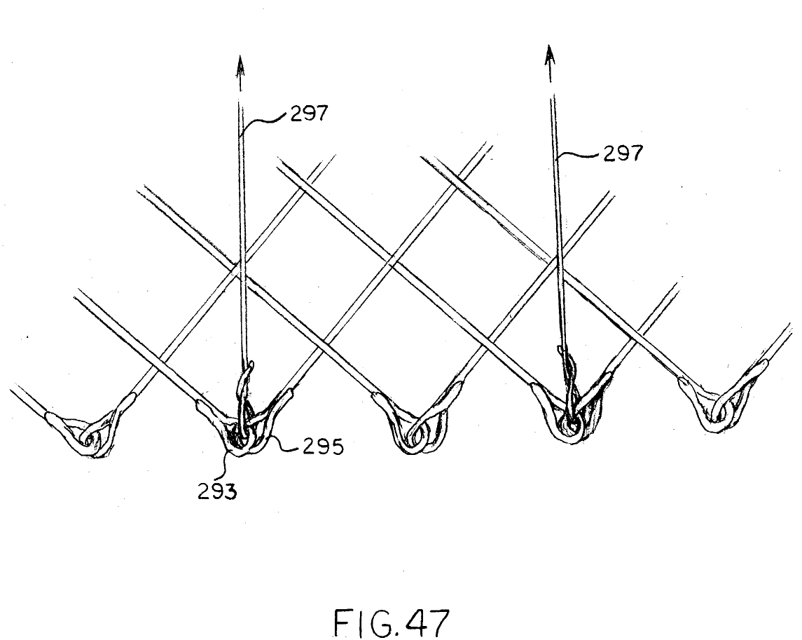

In the embodiment in FIG. 47, the intersecting wire ends at the distal end of tubular dilator 15/15B are each bent in opposite directions to form hooks 293 and 295, which are linked together as shown in FIG. 47. Hooks 293 and 295 can be formed by using the eye of a needle to bend each wire end. Pulling wires 297 are attached at four of these links by tying a knot. This linked arrangement will prevent the unraveling of tubular dilator 15/15B and will not interfere with the radial expansion of tubular dilator 15/15B since hooks 293 and 295 can freely rotate with respect to one another.

FIGS. 48-52 illustrate another method of preventing the unraveling of the open-ended wire matrix at the distal rim of tubular dilator 15/15B. This is accomplished by installing flexible hinges 299 at each wire intersection in the mesh near the distal rim of tubular dilator 15/15B which do not impede its radial expansion. Hinges 299 are produced by dipping the distal end of tubular dilator 15/15B into an appropriate liquid elastic material such as latex, Permalume, polyethylene C-flex, silicone rubber, or the like, with a strong adhesive property. To prevent the filming of the elastomer on the wire mesh, it should be diluted and the dipping and drying should be repeated several times. The elastomer will be driven by surface tension to the wire intersections, where it will gradually accumulate to form flexible hinges 299. The viscosity of the elastomer can also be reduced by raising its temperature. Alternately, the film of the elastomer can be removed by blowing with an air jet. Another approach of depositing elastomer at each wire intersection is through using a small pipette to place individual droplets 301 (FIG. 52).

The optional use of flare 16 at the distal end of tubular dilator 15, as shown in FIG. 1C, reduces the initial resistance of the stretched out braided dilator to the axial compression force applied to it by tension on pulling wires 20a and 20b, which is in addition to the resistance stemming from the tissue being dilated in the percutaneous access channel. In the stretched out initial state, each wire mesh of tubular dilator 15, having the shape of a small equilateral parallelogram, is aligned with its opposite sharp angles along the axial direction of the dilator. Consequently, when tubular dilator 15 is subjected to compression along the same axes, the kinematics of its geometry yields resistance to compression directed against these sharp angles. Such resistance, however, can be effectively reduced with the help of a small flare 16, formed at the distal end of the stretched out tubular dilator 15. This flare provides a gradient from sharp to flatter angles of the parallelograms' opposite axially-oriented angles, progressing towards the flare's rim, as shown in FIG. 1C. Funnel end 18 of tubular dilator 15 also provides a similar gradient, progressing towards the top rim of the funnel. As a result, both gradients become starting sites for triggering the compression of the whole stretched out braid from both sides of the abdominal wall, which in turn, generates its radial expansion and hence the radial dilation of the surrounding tissue as required in the formation of the percutaneous access port.

During dilation of the initial percutaneous channel passing across the wall of the body cavity, the distal stretched out portion of the tubular dilator 15/15B/251 extending into the body cavity expands more readily and to a larger diameter than the part constrained by the tissue of the wall. As a result, tubular dilator 15/15B/251 also functions as an expansion clamp 22, forming an hour glass shape as shown in FIG. 4, which firmly anchors tubular dilator 15/15B/251 along with percutaneous access device 64 to the wall of the body cavity (e.g. the abdominal wall) as shown in FIG. 10. If funnel connector 265 is used to attach dilator 15/15B/251 to percutaneous access device 64, this anchoring shape is better described as a bell as shown in FIGS. 38C and 41. Once expansion clamp 22 is formed, the narrow part of tubular dilator 15/15B/251, constrained within the percutaneous channel, will continue to radially expand as described above to the desired diameter and dilate the tissue, provided that there is an additional increase in the axial compression force to overcome tissue resistance.

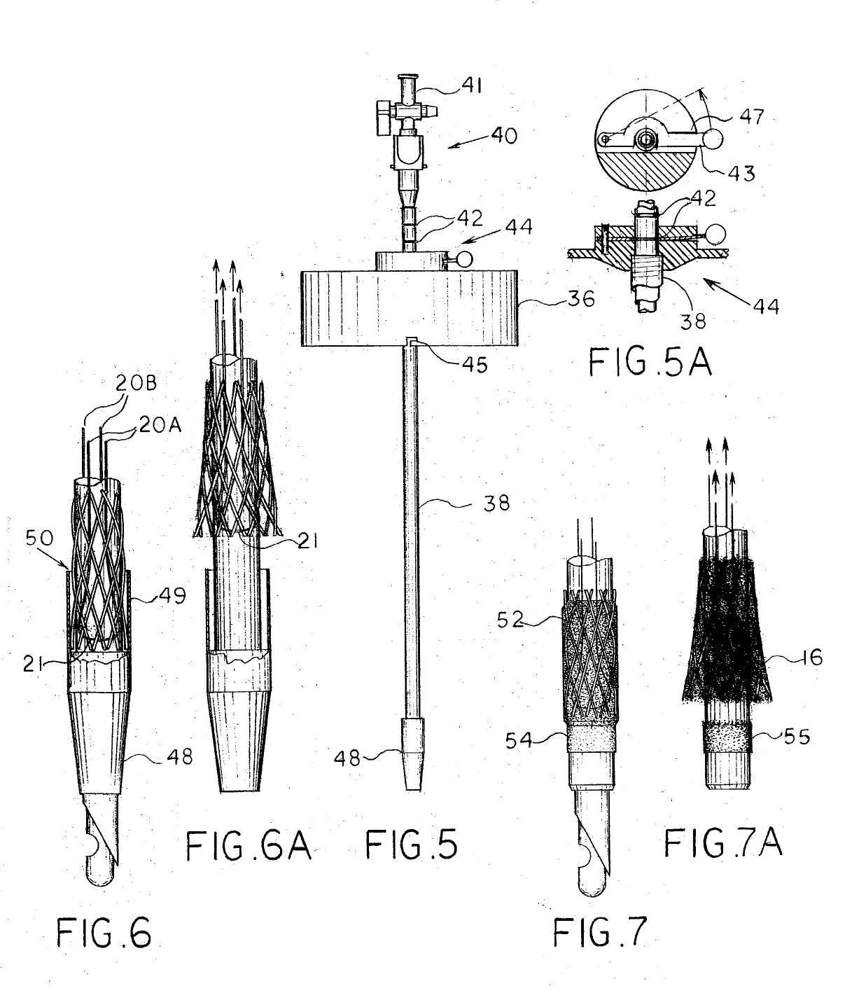

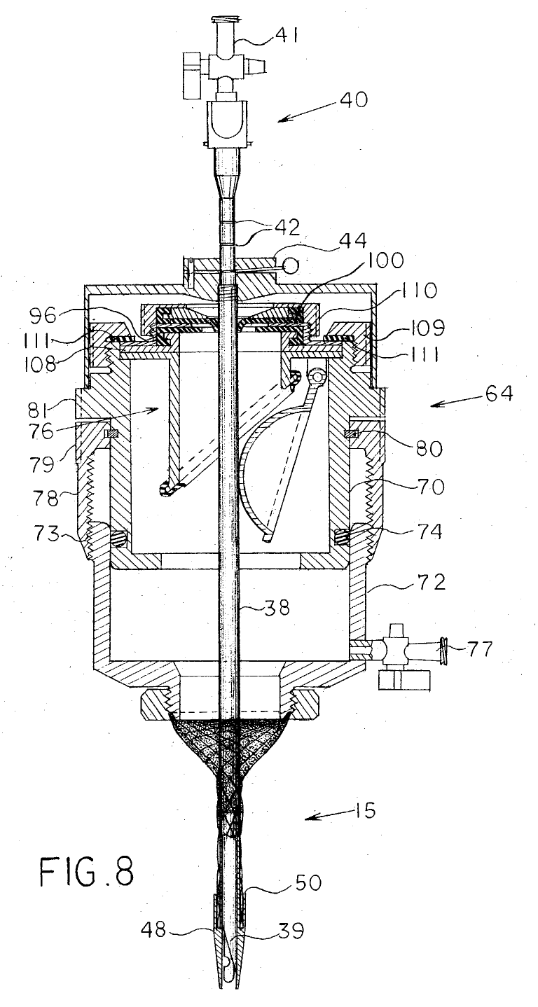

FIG. 5 is a perspective view of a housing cap 36 with a hollow stylet 38 mounted at its center, which slidably receives at its axial lumen an insufflation and access needle 40, which is of the Veress type. Stylet 38 along with needle 40 are initially held inside percutaneous access device 64 as shown in FIG. 8. Housing cap 36 has a pair of L-shaped catches 45 (shown for one side in FIG. 5) used to lock housing cap 36 on the proximal end of percutaneous access device 64. Needle 40 has an insufflation valve 41 mounted on its proximal end, used for initial insufflation, and a needle tip 39 at its distal end of the Veress type, having a spring-loaded obturator 48 for safety (shown in FIG. 8). Several notches 42 on the needle's proximal side and a spring-loaded stop 44 allow to preset the length of the needle advancement from the distal end of stylet 38 in order to match variations in abdominal wall thickness, and to prevent excessive or insufficient penetration of the needle into the abdominal cavity in laparascopic applications. Spring-loaded stop is shown in greater detail in FIG. 5A, and uses a flat spring 43 to lock with notches 42. A small ledge 47 on spring-loaded stop 44 is used to hold flat spring 43 in locked position. A small covering cap 48 at the distal end of stylet 38—also shown in an enlarged and partially sectional view in FIGS. 6-6A—is used to initially hold the distal end of tubular dilator 15/15B, which is inserted under covering cap 48 from its open proximal side 50. Proximal side 50 of covering cap 48 has thin walls 49 relative to the rest of the cap, forming an annular opening.

In FIG. 6, covering cap's thin walls 49 are shown partially in cross section in order to illustrate the attachment of pulling wires 20a and 20b to the distal end of tubular dilator 15/15B. The tapered end of covering cap 48 provides a smooth entry for the distal end of tubular dilator 15/15B into a percutaneous puncture. Thereafter, pulling wires 20a and 20b pull out in the proximal direction the end of tubular dilator 15/15B, thereby releasing it from covering cap 48 as shown in FIG. 6A and FIG. 9. By applying further tension on pulling wires 20a and 20b, tubular dilator 15/15B can be partially radially expanded to 6 or 8 mm. This will allow the removal of stylet 38 together with housing cap 36, along with the insufflation and access needle 40, thereby clearing the space of the axial lumen of tubular dilator 15/15B as shown in FIG. 10. Tubular dilator 15/15B can be further expanded by applying more tension on pulling wires 20a and 20b for the introduction of surgical instruments and fully installing percutaneous access device 64 in abdominal wall 32 in working position, shown in more detail in FIGS. 19-27.

FIGS. 7 and 7A show an alternate embodiment for mounting the distal end of tubular dilator 15/15B at the distal end of stylet 38, without using covering cap 48, which allows the reduction in diameter of the initial percutaneous puncture. Instead, a short piece of a thin-walled water soluble tubing 52 is positioned over the distal end of tubular dilator 15/15B, containing both pulling wire loops attached to the rim of tubular dilator 15/15B. The leading end 54 of tubing 52 goes beyond the rim of tubular dilator 15 and is firmly glued to the surface of stylet 38. Water soluble tubing 52 can be made of, for example, the same gelatinous non-allergenic material from which capsules are made for medical use to hold medications. Once inserted through the percutaneous puncture and in contact with blood, tubing 52 will rapidly absorb water and become soft, thereby releasing the distal end of tubular dilator 15/15B. This releasing process can be accelerated by applying tension on pulling wires 20a and 20b, which tears tubular dilator 15 from the leading end 54 of tubing 52, leaving a separated end of the tubing 55 attached to the surface of stylet 38 as shown in FIG. 7A, and allows tubular dilator 15 to expand. Thereafter, stylet 38 together with housing cap 36 can be removed from the axial lumen of tubular dilator 15/15B, leaving percutaneous access device 64 fully installed, as already described above for the first embodiment shown in FIG. 6A. In order to protect tubing 52 from absorbing moisture during shelf storage of percutaneous access device 64, the tip of stylet 38 must be covered by a plastic storage cap containing a moisture absorbing agent (not shown), which should be removed shortly before the use of the device. As an alternate approach to tubing 52, a dissolvable glue can be used to directly attach the distal end of tubular dilator 15/15B to stylet 38 (not shown).

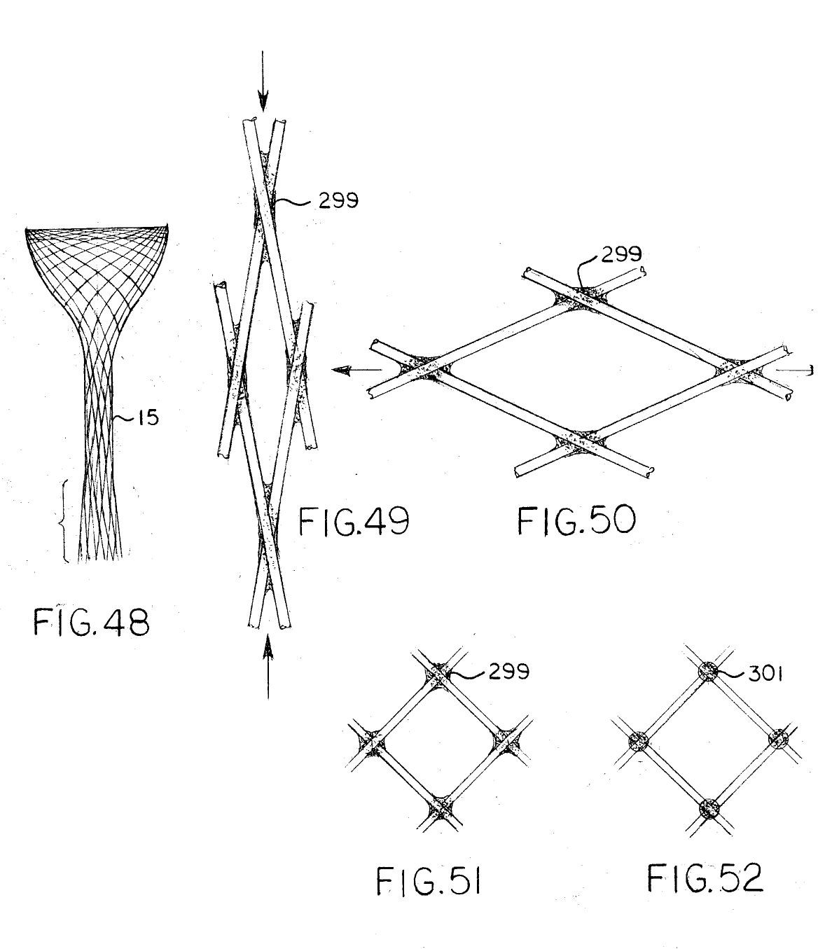

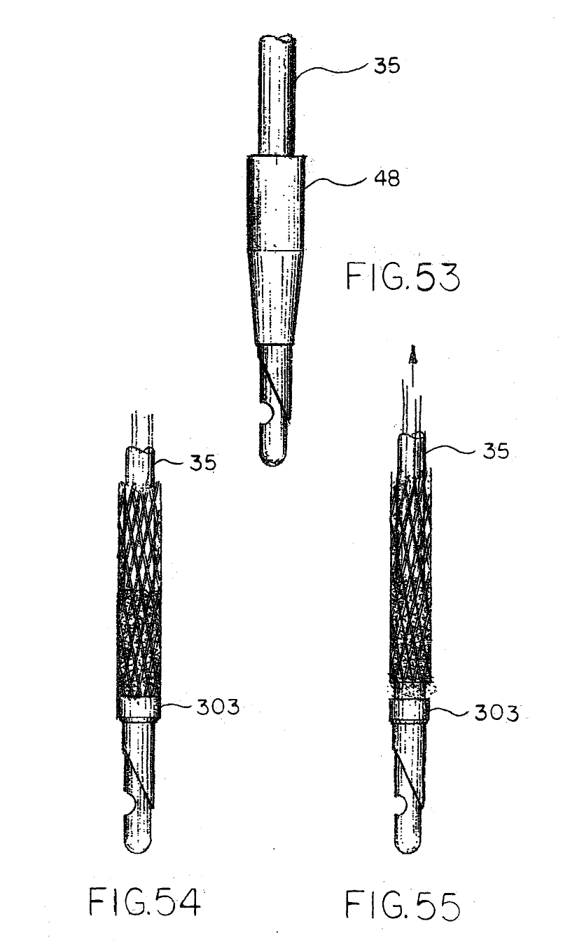

FIG. 53 shows an alternate approach without using stylet 38 to percutaneously deploy tubular dilator 15/15B/251, where covering cap 48 is directly attached to the distal end of a Veress needle 35. Veress needle 35 is directly inserted through housing cap 36 containing no stylet. The distal end of tubular dilator 15/15B/251 is similarly contained in the annular opening of covering cap 48. As an alternative to covering cap 48, tubing 52 or dissolvable glue can similarly be used to directly attach the distal end of tubular dilator 15/15B/251 to veress needle 35. As yet another alternative, in FIGS. 54-55, veress needle 35 has a tapered ring 303 near its distal end so that if the distal end of tubular dilator 15/15B/251 is directly glued to the needle surface above ring 303, tubular dilator 15/15B/251 will be flush with ring 303 providing a smooth continuous surface for percutaneous insertion. FIG. 55 shows the detachment of the glued tubular body 15/15B/251 from needle 35. The advantage of not using stylet 38 is a simplified device and a narrower initial percutaneous puncture. However the advantage of stylet 38 is that it may provide an additional margin of safety when creating the initial percutaneous puncture track with a veress needle.

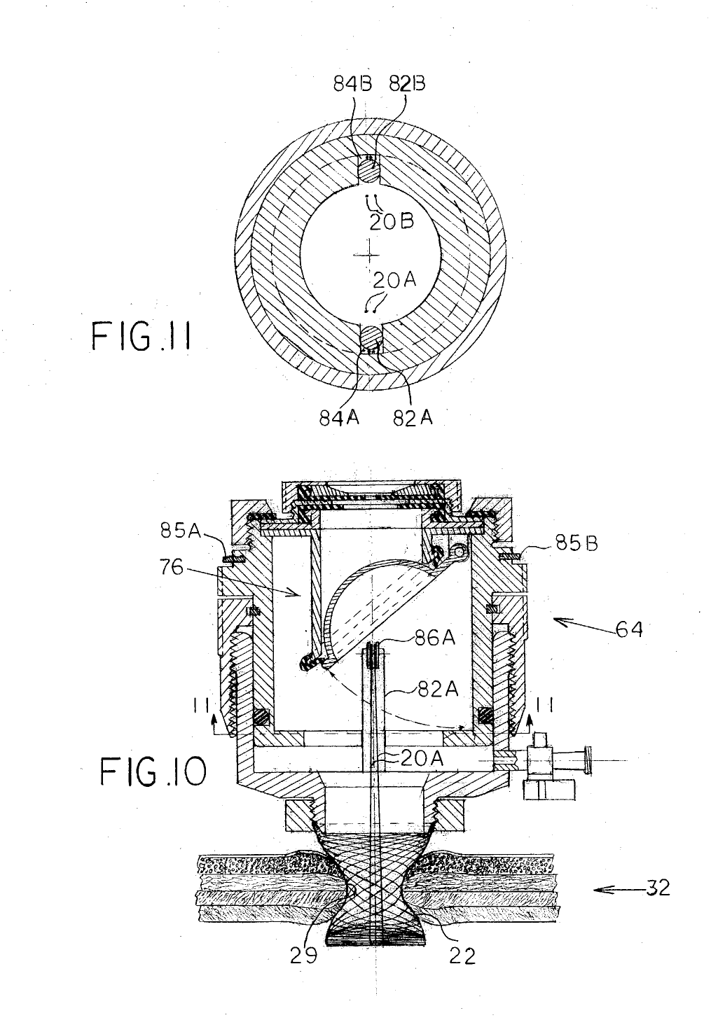

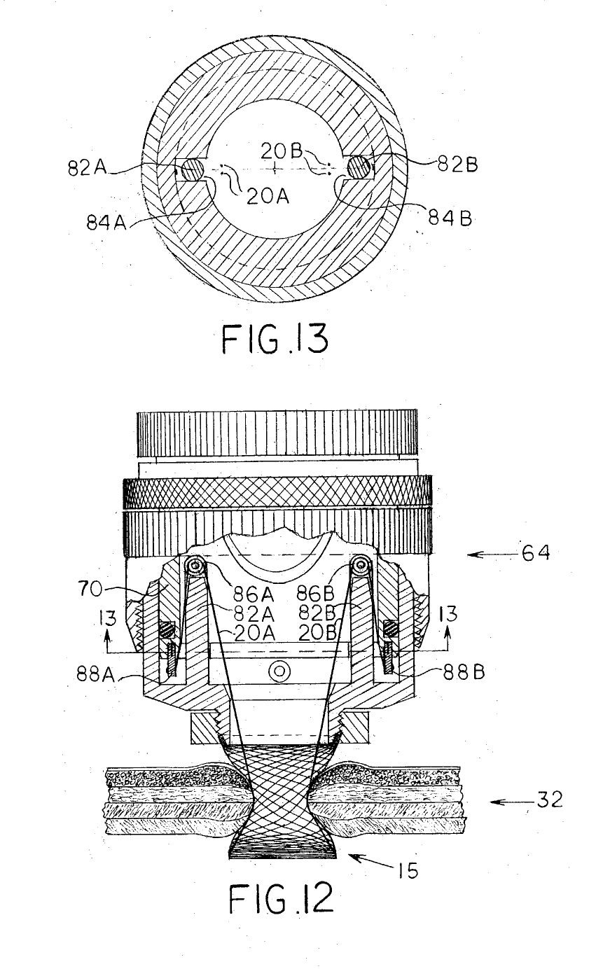

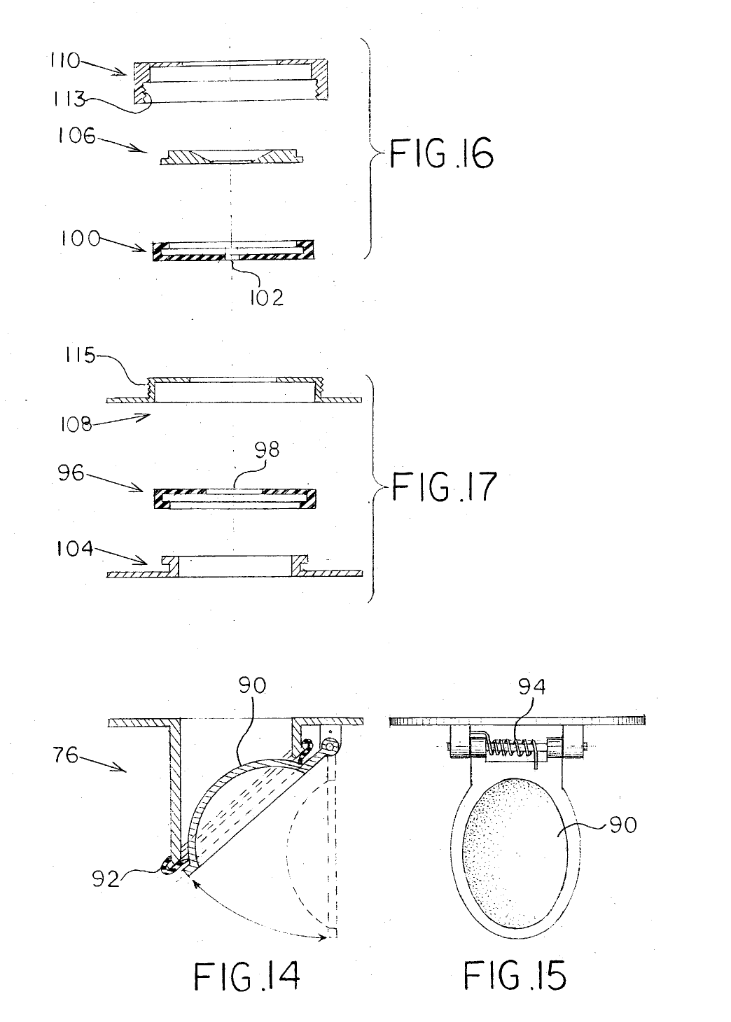

FIG. 8 shows the first embodiment of percutaneous access device 64 of the present invention, which integrates as follows. The device includes multifunctional tubular dilator 15/15B, stylet 38 which contains insufflation and access needle 40, a pneumostatic valve system 76 (for details see FIGS. 14-17), the mechanism for generating tension on pulling wires 20a and 20b for activating tubular dilator 15/15B (shown later in FIG. 12), and a secondary insufflation valve 77 for maintaining insufflation and for final releasing of insufflation gas. Percutaneous access device 64 is preferably made from a hard plastic, or other suitable material. This device could also use the laser cut tubular dilator 251 in place of wire braided tubular dilator 15/15B.

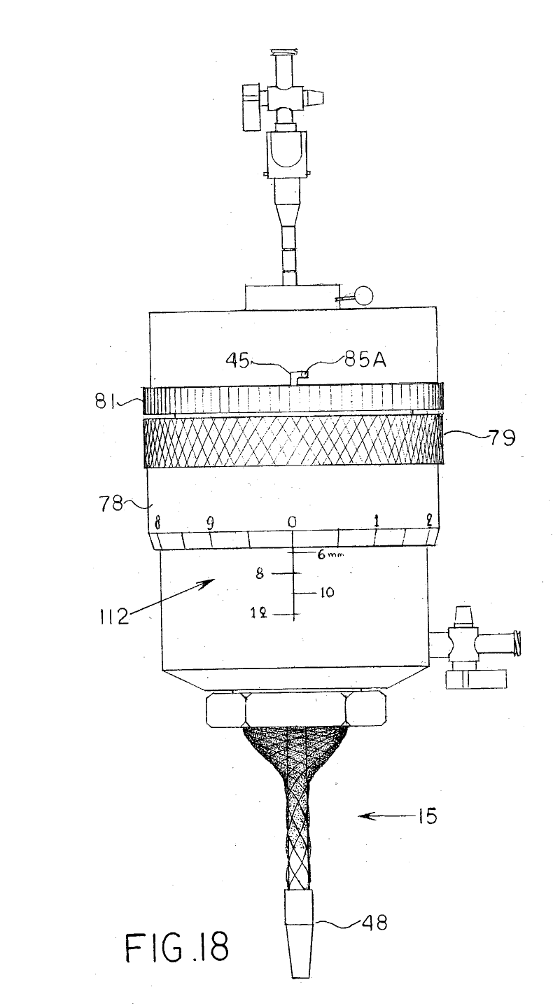

Percutaneous access device 64 generally has a cylindrical configuration and consists of two telescopically disposed cup-like parts, an inner part 70 and an outer part 72, fitted together with a large o-ring 74, providing the pneumostatic seal between them. The axial displacement of outer part 72 with respect to inner part 70 controls the tension on pulling wires 20a and 20b, and is achieved by manual rotation of a large knurled nut 78 engaged by its thread with a corresponding thread 73 on outer part 72, and attached by an internal retaining ring 80 to inner part 70. Inner part 70 has a knurled ring 81 on its outer surface, which is held by hand while nut 78 is turned. To prevent rotational slippage between parts 70 and 72, the larger outer part 72 is provided with two rods 82a and 82b as shown sectionally in FIG. 11, which slide axially within slats 84a and 84b at the bottom of inner part 70. Rods 82a and 82b are both equipped at their proximal ends with pulleys 86a and 86b as shown in FIG. 12, which pass pulling wires 20a and 20b over them. The distal ends of pulling wires 20a and 20b are attached to the distal rim of tubular dilator 15 by looping the wires as described previously. The proximal ends of pulling wires 20a and 20b are attached by means of small pins 88a and 88b (FIG. 12) to the bottom of inner part 70. Since pulling wires 20a and 20b are looped, both strands of each wire pass over their respective pulley as partially seen for pulling wire 20a in FIG. 10, and both strands are attached to their respective pin. The involvement of pulleys in this design allows tension on pulling wires 20a and 20b to be generated when parts 70 and 72 are moving toward each other and thereby reducing the overall length of percutaneous access device 64, which makes it more convenient to operate during the surgical procedure. Inner part 70 has a pair of pins 85a and 85b extending from its surface on opposite sides (FIG. 10), which engage with L-shaped catches 45 on housing cap 36 to initially lock housing cap 36 along with stylet 38 in percutaneous access device 64, as shown in a perspective view for one catch in FIG. 18.

FIG.9 shows the same percutaneous access device 64 as in FIG. 8, after stylet 38 with covering cap 48 have fully penetrated abdominal wall 32 creating percutaneous channel 29, and the distal end of tubular dilator 15/15B has been released from covering cap 48 by means of tension on pulling wires 20a and 20b. At this step, insufflation and access needle 40 is completely retracted into stylet 38.

FIGS. 14-17 show in detail pneumostatic valve system 76, also shown inside percutaneous access device 64 in FIGS. 8-10. FIGS. 14-15 show a flapper 90 of the valve system, which instead of being flat as in a conventional valve, is ellipsoidal. This shape helps prevent the catching of a tissue sample by the front edge of flapper 90, by deflecting the flapper from the axial passage way while an instrument, such as a tissue extractor, is being pulled out from percutaneous access device 64. Flapper 90 is spring-loaded by a helical spring 94 which presses flapper 90 against a rubber gasket 92, making an air tight seal and keeping the valve completely sealed when there is no instrument inside percutaneous access device 64. The proximal part of valve system 76, shown in FIGS. 16 and 17 is not much different from conventional valves. It consists of two sets of rubber gasket seals and plastic holders, one for larger diameter surgical instruments (FIG. 17), which is permanently installed in percutaneous access device 64, and a removable part (FIG. 16), which is used for instruments with smaller diameters. Each of these sets consists of one rubber gasket 96 with a central opening 98 for larger instruments, and a rubber gasket 100 with a smaller central opening 102 for smaller instruments. Each set has one metal or plastic holder 104 and 106 over which these gaskets are stretched to hold them in functional position. Each set also has a cap 108 and 110 for covering the gaskets, and for attaching them to percutaneous access device 64, shown sectionally in FIG. 8. These gaskets provide a gas seal while an instrument is inserted into percutaneous access device 64, and flapper 90 is open. Cap 108 is permanently attached to inner part 70 of percutaneous access device 64 by a nut 109 mounted at the proximal end of inner part 70, along with a gasket 111 to provide a gas seal for this nut. Cap 110 is removably attached by a nut 113 to a thread 115 on cap 108.

FIG. 18 shows an enlarged perspective view of percutaneous access device 64 in a closed initial position. Nut 78 has knurled ring 79 mounted on its surface for the easier turning and activation of tubular dilator 15. In order to expand tubular dilator 15 to the desired diameter, numbers 112 on the surface of percutaneous access device 64, expressed in millimeters, are provided, much like a micrometer scale. By turning nut 78, inner part 70 of percutaneous access device 64 moves upwards or downwards, contracting or expanding the length of the entire device, and radially contracting or expanding tubular dilator 15 by the amount corresponding to numbers 112.

The use of tubular dilator 15/15B/251 as a tissue dilator and anchor may also be applied to the distal end of long cannulas introduced through a primary percutaneous access port of the same character, thus providing a secondary access port extending into internal hollow organs for introduction of diagnostic and treatment devices (not shown).

FIGS. 19-27 show the complete operational sequence of using percutaneous access device 64 during a surgical procedure in forming a working percutaneous access channel.

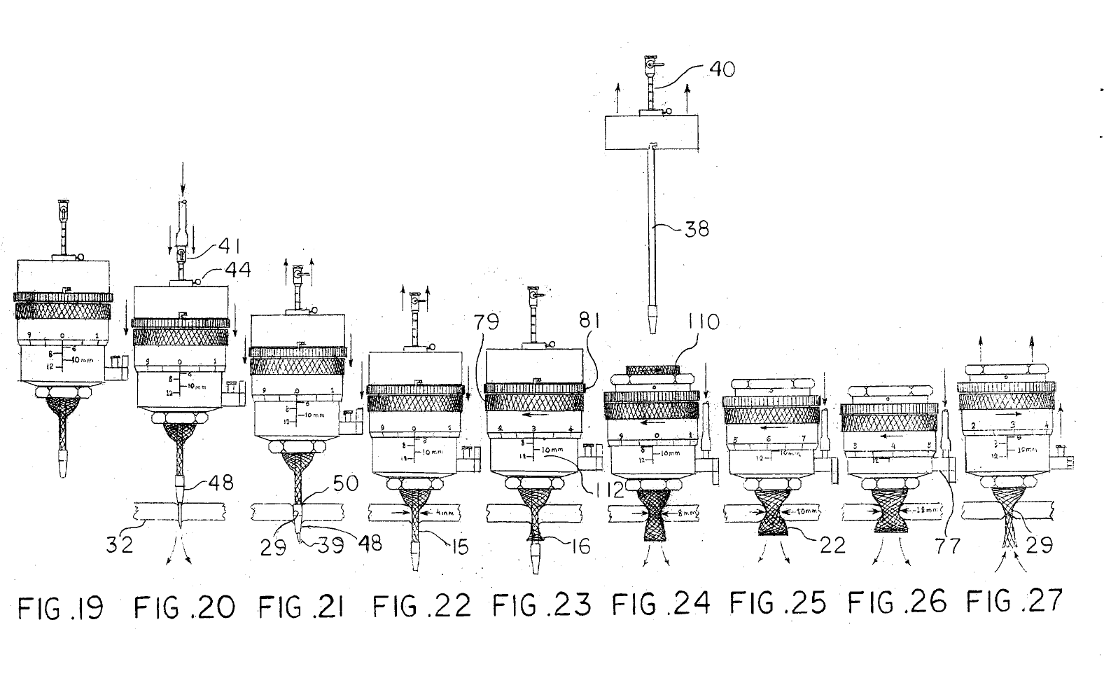

The method of using percutaneous access device 64 requires first measuring the thickness of abdominal wall 32 (in laparascopic applications) for appropriately presetting the length of the advancement of needle 40 from stylet 38. Needle 40 is advanced using spring-loaded stop 44. At this point, a special new external device for the non-invasive lifting of the abdominal wall can be optionally applied on the abdomen at the surgical site in order to provide a small clearance in the abdomen for safer initial needle insertion. Once needle 40 has been advanced, percutaneous access device 64 is moved forward until needle 40 passes through abdominal wall 32, and the tip of covering cap 48 touches the skin, as shown in FIG. 20. This positions the needle correctly for insufflation through insufflation valve 41. This step (FIG. 20) is not performed if using the embodiment where covering cap 48 is directly attached to needle 40 without using stylet 38 as previously described, in which case insufflation takes place in the step shown in FIG. 21. After insufflation of the abdomen, needle 40 is partially retracted back into stylet 38 so that its needle tip 39 still remains outside covering cap 48 and within the puncture track. Next, needle tip 39 along with covering cap 48 are both pushed forward through the same track until proximal end 50 of covering cap 48 is flush with the skin as shown in FIG. 21, which creates the initial percutaneous channel 29. At this point, needle 40 is completely retracted into stylet 38 for safety, and covering cap 48 is further pushed into the abdominal cavity until the base of the funnel of tubular dilator 15/15B is flush with the skin as shown in FIG. 22. Now, tubular dilator 15/15B is correctly positioned for radial dilation of percutaneous channel 29. Next, the distal end of tubular dilator 15 is released from proximal side 50 of covering cap 48, so that the dilator can be expanded (FIG. 23). This release and expansion is achieved by turning knurled ring 79 (which turns nut 78) positioned around the housing of percutaneous access device 64 to approximately the 6 mm point of numbers 112, causing pulling wires 20a and 20b to pull the distal end of tubular dilator 15 out from covering cap 48 as shown in FIG. 23. Turning knurled ring 79 further causes pulling wires 20a and 20b to apply more axial compression, thus radially expanding tubular dilator 15/15B more, and allowing stylet 38 together with needle 40 to be removed from the dilator, and entirely from percutaneous access device 64 as shown in FIG. 24. Knurled ring 79 is turned while holding knurled ring 81, which is part of the device housing. To complete formation of percutaneous channel 29, the stem part of tubular dilator 15/15B is radially expanded further, thereby dilating the channel to a desired size as shown in FIGS. 24-26, allowing the required surgical instruments to fit through the device and channel. At this point, expansion clamp 22 is also formed at the distal end of tubular dilator 15/15B, firmly anchoring the dilator along with percutaneous access device 64 in place throughout the procedure. During the procedure, if additional insufflation pressure is needed, gas is administered through secondary insufflation valve 77. Depending on the size of the surgical instruments needed, cap 110 with smaller rubber gasket 100 can be unscrewed from the top of percutaneous access device 64 (FIGS. 24-25), allowing larger diameter instruments to be inserted. At the end of the surgical procedure and after removal of all surgical instruments, gas is released from valve 77. Then knurled ring 79 is turned completely in the opposite direction to collapse tubular dilator 15/15B to its initial width as shown in FIG. 27, allowing percutaneous access device 64 along with tubular dilator 15 to be safely removed from percutaneous channel 29.

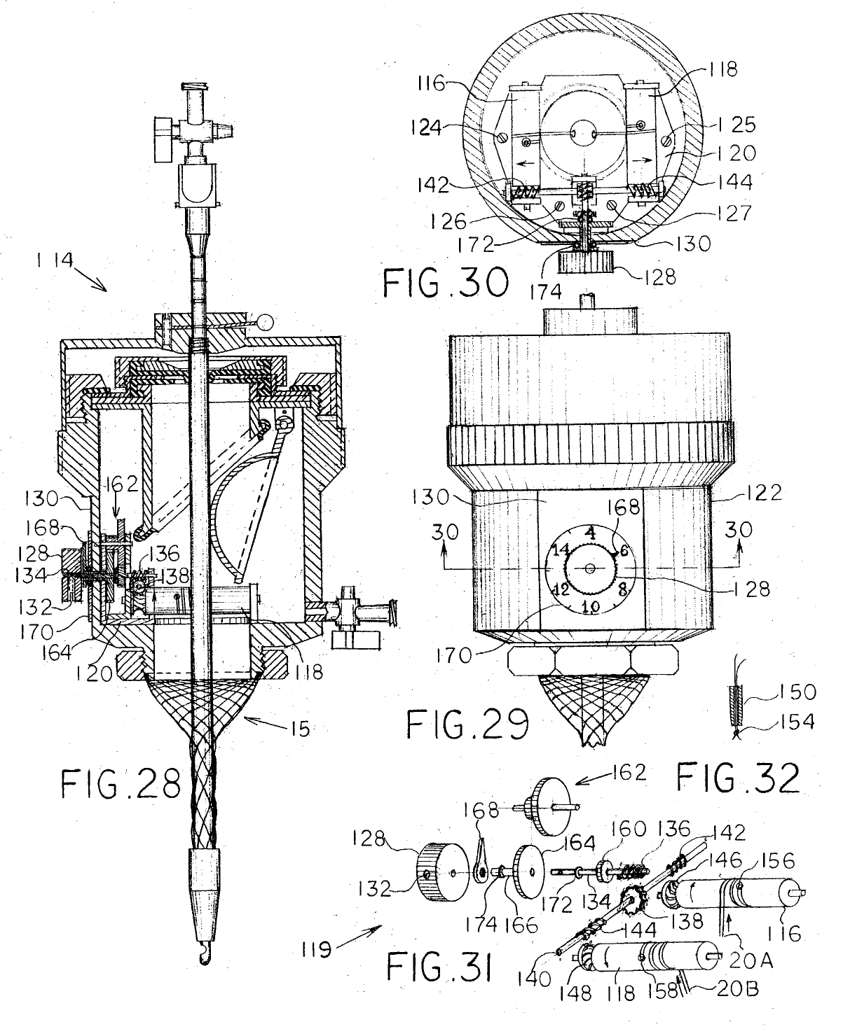

Illustrated in FIGS. 28 through 32 is a second embodiment of a percutaneous access device 114 of the present invention. It employs a different mechanism for the axial compression of tubular dilator 15/15B than that disclosed in the first embodiment of this device, but is otherwise similar. While still utilizing the same pulling wires 20a and 20b and attachment method to tubular dilator 15/15B as in the first embodiment, the pulling action is accomplished differently by winding these wires around two rollers 116 and 118 as shown in FIGS. 28, 30, and 31, which turn in opposite directions. In FIG. 31, a gear train 119 for manual activation of these rollers is shown in an exploded view. Gear train 119 is mounted on a metal chassis 120 as a separate module as shown in FIG. 30, having several brackets for holding gears. Chassis 120 is installed at the bottom of a cylindrical device housing 122 (FIG. 28), to which it is attached by several screws 124-127 (FIG. 30). An activation knob 128 for this gear train mechanism is positioned on a flat surface 130 cut from a portion of cylindrical device housing 122 as shown in FIG. 29. Knob 128 is attached by a set screw 132 to a driving shaft 134, having a worm gear 136 at its end which is meshed with a corresponding gear 138 attached to another shaft 140 positioned at 90 degrees to driving shaft 134 (FIG. 31). Shaft 140 has on it right and left hand gears 142 and 144, which are meshed with corresponding gears 146 and 148 attached to rollers 116 and 118 for turning them in opposite directions, which in turn, winds up or down pulling wires 20a and 20b. The ends of each pair of pulling wires 20a and 20b are attached to rollers 116 and 118 by means of tubular pins 150 and 152 (shown for one side in FIG. 32), through which each pair of wire ends is fed then tightened into a knot 154. Thereafter pins 150 and 152 are inserted into a corresponding hole 156 and 158 in rollers 116 and 118. Driving shaft 134 has a small gear 160 attached to it, which is meshed with a reduction gear 162, which in turn is meshed with a gear 164 which has a tubular shaft 166 coaxially arranged with driving shaft 134 and having at its end a pointer 168. Concentric with driving shaft 134 and pointer 168 is a position dial 170 on flat surface 130, as shown in FIGS. 28 and 29. Position dial 170 displays measurements which translates the manual rotation of knob 128 into the extent of radial dilation of tubular dilator 15 expressed in millimeters of its diameter. The pneumostatic seal for gear train 119 is provided by two small o-rings 172 and 174, the first one of which is positioned inside the percutaneous access device 114 on driving shaft 134, and the second positioned on tubular shaft 166 outside the percutaneous access device 114 as shown in FIGS. 30-31.

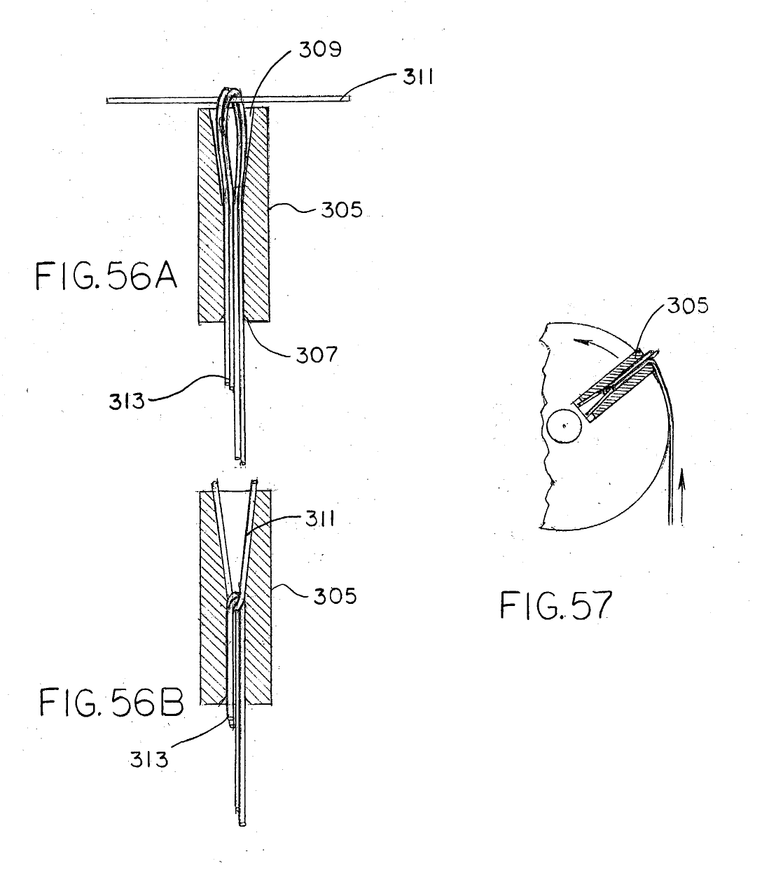

FIGS. 56-57 show an alternate way of securing the ends of the pairs of pulling wires inside pins. The wire ends 313 of the pulling wires are attached to a hollow metal pin 305, which has a tubular opening 307 on one side, and a funnel-shaped opening 309 on the other. The wire ends are threaded through tubular opening 307, and then looped around and threaded back through funnel-shaped opening 309. The wire ends are secured inside metal pin 305 by means of a short wire 311, about one inch long and 0.005 inches in diameter, which is inserted under the wire ends at the point where they loop back through funnel-shaped opening 309. The wire ends are then pulled to the bottom of funnel-shaped opening 309, which fixes them tightly by driving them in a wedge along with wire 311 as shown in FIG. 56B, thus providing secure attachment.

The operation of percutaneous access device 114 (FIGS. 28-32) is very similar to operating percutaneous access device 64 of the first embodiment as shown in FIGS. 19-27, with the difference being that knob 128 is turned instead of nut 78 to activate tubular dilator 15/15B. An advantage of this embodiment is that its housing is comprised of a single part, which stays the same short length throughout the procedure, unlike the previous embodiment where telescopic motion is utilized between two main parts, changing the length of the entire housing.

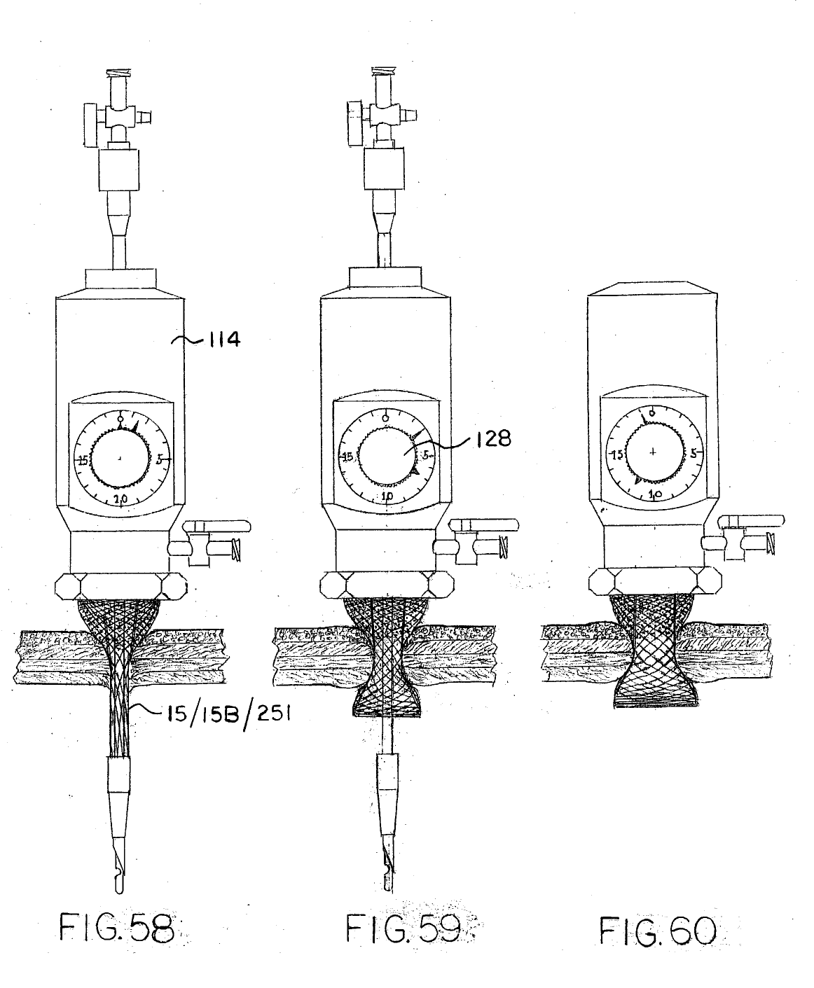

FIGS. 58-60 show a variation of percutaneous access device 114 utilizing a similar gear train 119 and knob 128, but in an overall narrower housing. This is achieved by using an alternate narrower valve system, many of which are commercially available for laparoscopic applications.

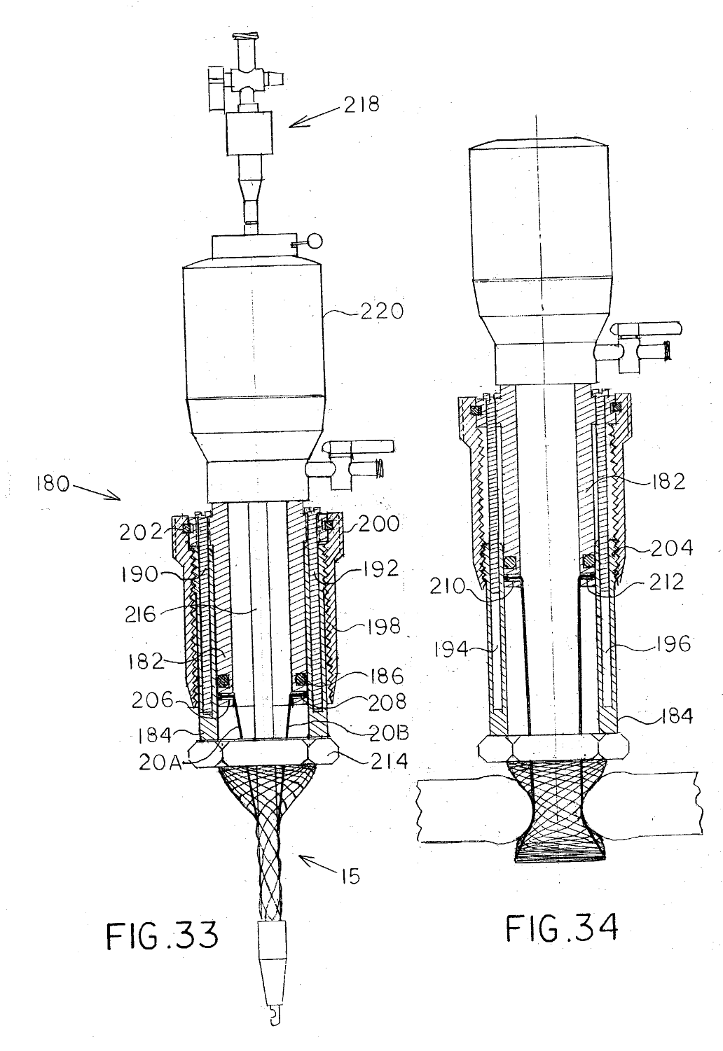

Illustrated in FIGS. 33 through 35 is a third embodiment of a percutaneous access device 180 of the present invention. This embodiment uses a simpler mechanism for placing tension on the same pulling wires 20a and 20b to activate tubular dilator 15/15B. The device housing is comprised of two telescopically moving cylindrical parts, an inner part 182, slidably received in an outer part 184. The distal end of inner part 182 houses a rubber gasket 186 in a circular slot to provide a gas seal between inner part 182 and outer part 184. Inner part 182 has sliding rods 190 and 192 contained and moving within channels 194 and 196 in outer part 184. This rod and channel arrangement prevents inner part 182 and outer part 184 from rotating with respect to each other. A nut 198 with a knurled ring 200 provides the top half of the external housing of percutaneous access device 180. Nut 198 is attached to inner part 182 by a retaining ring 202, which allows only rotational motion of the nut in place. Nut 198 is engaged with a thread 204 on outer part 184, so that when nut 198 is turned, inner part 182 moves telescopically with respect to outer part 184. Both strands of each pulling wire 20a and 20b are attached by tubular pins 206 and 208 to the distal end of inner part 182 into holes 210 and 212. Tubular pins 206 and 208, and the method of fastening the wire ends within them are similar to what is shown in FIG. 32 for the second embodiment. The other ends of pulling wires 20a and 20b are identically attached to the distal end of tubular dilator 15/15B, as in the first two embodiments. Tubular dilator 15/15B is similarly attached to the distal end of outer part 184 with an attachment nut 214. A stylet 216 containing an insufflation and access needle 218 is housed within the axial lumen of inner part 182. This stylet and needle assembly is very similar to the one used in the previous two embodiments, including utilizing a similar spring-loaded catch mechanism to advance the needle as shown in FIG. 5A. The difference is that no covering cap 36 is employed to mount the stylet to the device, and its length may differ. The distal end of tubular dilator 15/15B is contained within the proximal side of a covering cap at the distal end of stylet 216, identical to FIGS. 5-7 in the first embodiment.

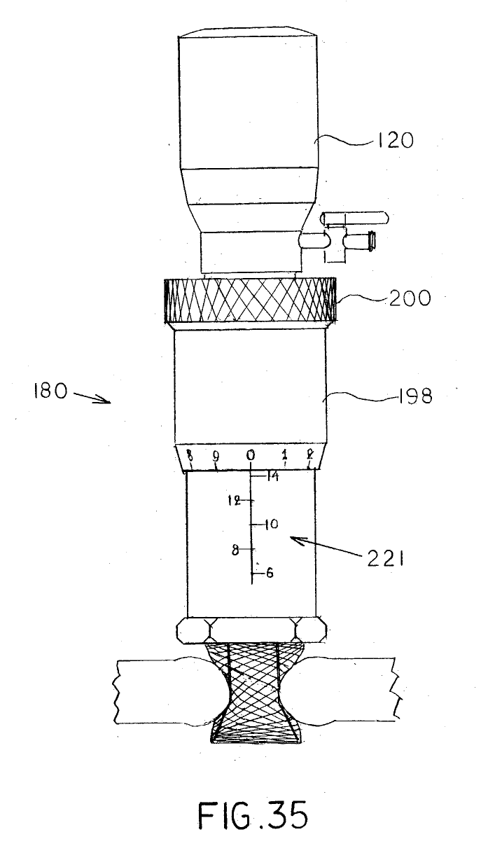

Percutaneous access device 180 does not utilize the pneumostatic valve system 76 as in the first two embodiments. Instead, a commercially available standard insufflation valve 220 is used, such as those employed in the Innerdyne Step devices. Valve 220 is attached to the proximal end of inner part 182, with the needle and stylet assembly passing through it. As shown in FIG. 35, the external housing of outer part 184 displays a scale 221, expressed in millimeters, corresponding to the amount of radial expansion of tubular dilator 15/15B. When nut 198 is turned so that inner and outer parts 182 and 184 move apart and the entire device housing elongates, tension is placed on pulling wires 20a and 20b, and tubular dilator 15/15B expands as shown in FIG. 34. When nut 198 is turned the other way, the parts move together and tubular dilator 15/15B contracts. Nut 198 is turned while holding the stationary housing of valve 220. This embodiment provides the advantage of being simpler in design and narrower in width. Operating and installing percutaneous access device 180 in abdominal wall 32 is very similar to the procedural steps shown for the first embodiment in FIGS. 19-27.

In FIGS. 61-64, a sheath 317 is used to initially cover the entire tubular dilator 15/15B/251 when attached to and deployed with the insufflation and access needle. Sheath 317 can be made from thin flexible polymeric material such as polyethylene, tetrafluoroethylene, or the like. It can be glued to tubular dilator 15/15B/251 or vacuum sealed to it for example. Sheath 317 has threads 319 and 321 attached inside at its distal end. Threads 319 and 321 have loops 323 and 325 for pulling with a finger. Near the distal attachment points of threads 319 and 321 to sheath 317 are formed small notches 327 and 329 to slightly weaken the sheath so that when threads 319 and 321 are pulled, sheath 317 will separate and open from its distal end as shown in FIG. 62. After removal of the insufflation and access needle as shown in FIG. 63, sheath 317 is left in place on tubular dilator 15/15B/251 during the surgical procedure, and removed along with the dilator as the dilator is withdrawn at the end. Sheath 317 need not be removed from the dilator. Sheath 317 functions to tightly keep tubular dilator 15/15B/251 constrained on the insufflation and access needle during percutaneous puncture for a narrow and smooth insertion.

1. A device for creating a percutaneous access port of variable size through dilation of an initial percutaneous penetration in a body cavity wall, said device comprising an insufflation and access needle for producing an initial puncture within the body cavity wall, a tubular dilator having an axial length, a distal end for insertion within said body cavity wall and a proximal end for extending outside of said body cavity wall during creation of said percutaneous access port, said tubular dilator comprising a tubular body having a stem section between said distal and proximal ends and having an expanded funnel-shape surface proximate its proximal end, means for releasably attaching the distal end of said tubular dilator to said insufflation and access needle, axial compression means for removably supporting said insufflation and access needle, said axial compression means being fixedly engaged to the proximal end of said tubular dilator and including means for creating an axial compressive force upon said tubular dilator to vary the axial length of said tubular dilator resulting in selective radial dilation thereof.

2. The device of claim 1 wherein said insufflation and access needle is provided with a stylet and stylet cover for selectively exposing said needle beyond said cover, said distal end of said tubular dilator being releasably secured to said stylet during said initial puncture of said cavity wall.

3. The device of claim 2 wherein the distal end of said tubular dilator is releasably frictionally secured between said stylet and stylet cover during said initial puncture of said cavity wall.

4. The device of claim 2 wherein the distal end of said tubular dilator is releasably secured to said stylet cover through the use of a water dissolvable adhesive.

5. The device of claim 1 wherein said axial compressive means comprises an inner housing and outer housing and at least two pulleys being positioned to engage at least two wire loops appended at their looped extremities to the distal end of said tubular dilator and at their non-looped extremities to said inner housing of said axial compression means such that as said inner and outer housings rotate with respect to one another, said at least two wire loops are caused to pass over said pulleys to initially release the distal ends of the tubular dilator from said insufflation and access needle and to selectively change the axial length of said tubular dilator.

6. The device of claim 1 wherein said tubular dilator is comprised of a member selected from the group consisting of braided stainless steel mono filaments, braided nitinol mono filaments and polymeric mono filaments creating a mesh of equilateral parallelograms displaying force-amplifying behavior as said dilator changes shape during axial compression.

7. The device of claim 6 wherein said mono filaments are approximately from 0.005 to 0.015 inches in diameter.

8. The device of claim 6 wherein said tubular dilator is approximately 50-75 mm in length and its stem section approximately 3.5 mm in diameter.

9. The device of claim 1 wherein said tubular dilator is provided with an expanded diameter with respect to said stem section prior to axial compression of said tubular dilator.

10. The device of claim 1 wherein said expanded funnel-shaped surface is coated with an elastic material.

11. The device of claim 10 wherein further elastic material is coated on the proximal end of said tubular dilator to act as a sealing gasket to said axial compression means.

12. The device of claim 1 wherein said tubular dilator is approximately 3.5 mm in diameter at its stem section when uncompressed and approximately 12 to 14 mm in diameter at its stem section when axially compressed.

13. The device of claim 5 wherein said wire loops are comprised of a member selected from the group consisting of high tensile strength titanium and high tensile strength stainless steel.

14. The device of claim 13 wherein said wire loops are approximately between 0.005″ and 0.010″ in diameter.

15. The device of claim 1 wherein said tubular dilator further comprises at least two wire loops appended at their looped extremities proximate the distal end of said tubular dilator such that in drawing said wire loops axially toward the proximal end of said tubular dilator said tubular dilator is caused to axially contract and radially expand.

16. The device of claim 15 wherein the looped extremities of said at least two wire loops are positioned so as to be threaded outwardly through at least two wire mesh parallelograms located at said distal end of said tubular dilator.

17. The device of claim 16 wherein each of said looped extremities is composed of wires threaded through said tubular dilator and separated by one mesh parallelogram.

18. The device of claim 1 wherein ribbon is fixedly appended to said tubular dilator and said means for creating an axial compressive force is appended to said ribbon for axially drawing said distal end to said proximal end of said tubular dilator.

19. The device of claim 18 wherein said ribbon is U-shaped passing over the distal end of said tubular dilator and having openings therein to receive said wire loops passing from within said tubular dilator to a position outside of said ribbon.

20. The device of claim 1 wherein said tubular dilator is comprised of a tube which has been laser cut to create a matrix of connected parallelograms, weakened at their joints to facilitate radial expansion upon its axial compression.

21. The device of claim 6 wherein said braided mono filaments are provided with wire ends at the distal end of said tubular dilator in which adjacent ends are bent in opposite directions to another to form a series of hooks.

22. The device of claim 21 wherein at least four wires are attached to said series of hooks such that in drawing said wires axially toward the proximal end of said tubular dilator causes said tubular dilator to axially contract and radially expand.

23. The device of claim 6 wherein said strands of mono filaments create points of intersection to provided said equilateral parallelograms, said points of intersection proximate the distal end of said tubular dilator being provided with flexible hinges.

24. The device of claim 5 wherein said inner housing and outer housing are reach cup-shaped, having an o-ring seal between them, said inner and outer housings joined by threaded engagement enabling the inner and outer housings to move axially with respect to one another.

25. The device of claim 1 wherein said axial compression means further comprises an inner volume, a longitudinal axis, a passageway for receiving instruments along said longitudinal axis and a valve for selectively maintaining an airtight seal within said inner volume.

26. The device of claim 25 wherein said valve comprises a flapper valve that is spring biased in a closed position whenever instruments are not being introduced to said inner volume.

27. The device of claim 1 wherein said axially compression means further comprises an insufflation valve for maintaining insufflation gas pressure and for selectively releasing said insufflation gas pressure.

28. The device of claim 1 wherein said axial compression means comprises a housing, a pair of rollers rotatably supported by said housing, each of which is positioned to engage one of at least two wire loops appended at their looped extremities to the distal end of said tubular dilator and at their non-looped extremities to said rollers and a control knob positioned external to said housing and having a shaft passing within said housing such that rotation of said control knob causes rotation of said rollers resulting in said at least two wire loops being caused to initially release the distal end of said tubular dilator from said insufflation and access needle and to selectively change the axial length.

29. The device of claim 28 wherein said axial compression means further comprises an inner volume, a longitudinal axis, a passage way for receiving instruments along said longitudinal axis and a valve for selectively maintaining an airtight seal within said inner volume.

30. The device of claim 29 wherein said valve comprises a flapper valve that is spring biased in a closed position whenever instruments are not being introduced to said inner volume.

31. The device of claim 28 wherein said axial compression means further comprises an insufflation valve for maintaining insufflation gas pressure and for selectively releasing said insufflation gas pressure.

32. The device of claim 1 wherein said axial compression means comprises a housing having inner and outer telescopically movable cylinders and a threaded nut engaging screw threads on said outer cylinder, the turning of which telescopically moves said inner and outer cylinders with respect to one another, tubular pins each for engaging one of the at least two wire loops appended at their looped extremities to the distal end of said tubular dilator and at their non-looped extremities to said tubular means such that rotation of said threaded nut causes telescopic movement between said inner and outer cylinders resulting in said at least two wire loops being caused to initially release the distal end of said tubular dilator from said insufflation and access needle and to selectively change the axial length of said tubular dilator.

33. The device of claim 32 wherein said outer cylinder is provided with channels and rods received therein for substantially resisting rotational movement between said inner and outer cylinders.

34. A device for creating a percutaneous access port of variable size through dilation of an initial percutaneous penetration in a body cavity wall, said device comprising an insufflation and access needle producing an initial puncture within the body cavity wall, a tubular dilator having an axial length, a distal end for insertion within said body cavity wall and a proximal end attached to a funnel-shaped connector, said funnel-shaped connector sized for connecting said tubular dilator to an axial compression means, means for releasably attaching the distal end of said tubular dilator to said insufflation and access needle and axial compression means for removably supporting said insufflation and access needle, said axial compression means including means for creating an axial compressive force upon said tubular dilator to vary the axial length of said tubular dilator resulting in selective radial dilation thereof.

35. The device of claim 34 wherein said tubular dilator is comprised of a member selected from the group consisting of stainless steel, nitinol and polymer.

36. The device of claim 35 wherein said axial compressive force and radial dilation are reversible.

37. The device of claim 34 wherein said funnel-shaped connector is comprised of rubber.

38. The device of claim 34 wherein a tapered ring is provided to the distal end of said insufflation and access needle proximate to the attachment of the distal end of said tubular dilator to said insufflation and access needle sized such that proximate said point of attachment, said tubular dilator is flush with said tapered ring.

39. The device of claim 1 wherein a tapered ring is provided to the distal end of said insufflation and access needle proximate to the attachment of the distal end of said tubular dilator to said insufflation and access needle sized such that proximate said point of attachment, said tubular dilator is flush with said tapered ring.

40. The device of claim 34 wherein said tubular dilator forms an anchor attachment within said body cavity wall while said tubular dilator is axially compressed.

41. The device of claim 1 wherein said tubular dilator forms an anchor attachment within said body cavity wall while said tubular dilator is axially compressed.

42. The device of claim 34 wherein said axial compression means further comprises an inner volume, a longitudinal axis, a passageway for receiving instruments along said longitudinal axis and a valve for selectively maintaining an airtight seal within said inner volume.

43. The device of claim 42 wherein said valve comprises a flapper valve that is spring biased in a closed position whenever instruments are not being introduced to said inner volume.

44. The device of claim 34 wherein said insufflation and access needle is provided with a stylet and stylet cover for selectively exposing said needle beyond said cover, said distal end of said tubular dilator being releasably secured to said stylet during said initial puncture of said cavity wall.

45. The device of claim 44 wherein the distal end of said tubular dilator is releasably frictionally secured between said stylet and stylet cover during said initial puncture of said cavity wall.

46. The device of claim 44 wherein the distal end of said tubular dilator is releasably secured to said stylet cover through the use of a water dissolvable adhesive.

47. The device of claim 34 wherein said axial compressive means comprises an inner housing and outer housing and at least two pulleys being positioned to engage at least two wire loops appended at their looped extremities to the distal end of said tubular dilator and at their non-looped extremities to said inner housing of said axial compression means such that as said inner and outer housings rotate with respect to one another, said at least two wire loops are caused to pass over said pulleys to initially release the distal ends of the tubular dilator from said insufflation and access needle and to selectively change the axial length of said tubular dilator.

48. The device of claim 34 wherein said tubular dilator is comprised of a member selected from the group consisting of braided stainless steel mono filaments, braided nitinol mono filaments and polymeric mono filaments creating a mesh of equilateral parallelograms displaying force-amplifying behavior as said dilator changes shape during axial compression.

49. The device of claim 48 wherein said mono filaments are approximately from 0.005 to 0.015 inches in diameter.

50. The device of claim 48 wherein said tubular dilator is approximately 50-75 mm in length and its stem section approximately 3.5 mm in diameter.

51. The device of claim 34 wherein said tubular dilator is provided with an expanded diameter at its distal end with respect to said stem section prior to axial compression of said tubular dilator.

52. The device of claim 34 wherein said tubular dilator is approximately 3.5 mm in diameter at its stem section when uncompressed and approximately 12 to 14 mm in diameter at its stem section when axially compressed.

53. The device of claim 47 wherein said wire loops are comprised of a member selected from the group consisting of high tensile strength titanium and high tensile strength stainless steel.

54. The device of claim 53 wherein said wire loops are approximately between 0.005″ and 0.010″ in diameter.

55. The device of claim 34 wherein said tubular dilator further comprises at least two wire loops appended at their looped extremities proximate the distal end of said tubular dilator such that in drawing said wire loops axially toward the proximal end of said tubular dilator said tubular dilator is caused to axially contract and radially expand.

56. The device of claim 55 wherein the looped extremities of said at least two wire loops are positioned so as to be threaded outwardly through at least two wire mesh parallelograms located at said distal end of said tubular dilator.

57. The device of claim 56 wherein each of said looped extremities is composed of wires threaded through said tubular dilator and separated by one mesh parallelogram.

58. The device of claim 34 wherein ribbon is fixedly appended to said tubular dilator and said means for creating an axial compressive force is appended to said ribbon for axially drawing said distal end to said proximal end of said tubular dilator.

59. The device of claim 58 wherein said ribbon is U-shaped passing over the distal end of said tubular dilator and having openings therein to receive said wire loops passing from within said tubular dilator to a position outside of said ribbon.

60. The device of claim 34 wherein said tubular dilator is comprised of a tube which has been laser cut to create a matrix of connected parallelograms, weakened at their joints to facilitate radial expansion upon its axial compression.

61. The device of claim 48 wherein said braided mono filaments are provided with wire ends at the distal end of said tubular dilator in which adjacent ends are bent in opposite directions to another to form a series of hooks.

62. The device of claim 61 wherein at least four wires are attached to said series of hooks such that in drawing said wires axially toward the proximal end of said tubular dilator causes said tubular dilator to axially contract and radially expand.

63. The device of claim 48 wherein said strands of mono filaments create points of intersection to provided said equilateral parallelograms, said points of intersection proximate the distal end of said tubular dilator being provided with flexible hinges.

64. The device of claim 47 wherein said inner housing and outer housing are each cup-shaped, having an o-ring seal between them, said inner and outer housings joined by threaded engagement enabling the inner and outer housings to move axially with respect to one another.

65. The device of claim 47 wherein said axial compression means further comprises an inner volume, a longitudinal axis, a passageway for receiving instruments along said longitudinal axis and a valve for selectively maintaining an airtight seal within said inner volume.

66. The device of claim 65 wherein said valve comprises a flapper valve that is spring biased in a closed position whenever instruments are not being introduced to said inner volume.

67. The device of claim 47 wherein said axially compression means further comprises an insufflation valve for maintaining insufflation gas pressure and for selectively releasing said insufflation gas pressure.

68. The device of claim 34 wherein said axial compression means comprises a housing, a pair of rollers rotatably supported by said housing, each of which is positioned to engage one of least two wire loops appended at their looped extremities to the distal end of said tubular dilator and at their non-looped extremities to said rollers and a control knob positioned external to said housing and having a shaft passing within said housing such that rotation of said control knob causes rotation of said rollers resulting in said at least two wire loops being caused to initially release the distal end of said tubular dilator from said insufflation and access needle and to selectively change the axial length.

69. The device of claim 68 wherein said axial compression means further comprises an inner volume, a longitudinal axis, a passage way for receiving instruments along said longitudinal axis and a valve for selectively maintaining an airtight seal within said inner volume.

70. The device of claim 69 wherein said valve comprises a flapper valve that is spring biased in a closed position whenever instruments are not being introduced to said inner volume.

71. The device of claim 68 wherein said axial compression means further comprises an insufflation valve for maintaining insufflation gas pressure and for selectively releasing said insufflation gas pressure.

72. The device of claim 34 wherein said axial compression means comprises a housing having inner and outer telescopically movable cylinders and a threaded nut engaging screw threads on said outer cylinder, the turning of which telescopically moves said inner and outer cylinders with respect to one another, tubular pins each for engaging one of the at least two wire loops appended at their looped extremities to the distal end of said tubular dilator and at their non-looped extremities to said tubular means such that rotation of said threaded nut causes telescopic movement between said inner and outer cylinders resulting in said at least two wire loops being caused to initially release the distal end of said tubular dilator from said insufflation and access needle and to selectively change the axial length of said tubular dilator.

73. The device of claim 72 wherein said outer cylinder is provided with channels and rods received therein for substantially resisting rotational movement between said inner and outer cylinders.

74. A tubular dilator for insertion within and expansion of a percutaneous access port, said tubular dilator comprising a tube having been laser cut producing a matrix of connected parallelograms, weakened at their joints to provide for radial expansion upon axial compression of said tube.

75. The tubular dilator of claim 74 wherein said tube comprises a member selected from the group consisting of stainless steel and nitinol and alloys thereof.

76. The device of claim 6 wherein said axial compressive force and radial dilator are reversible.
